(19) United States
(12) Patent Application Publication
(10) Pub. No.: US 2025/0216349 A1
(43) Pub. Date: Jul. 3, 2025

(54) SENSOR ARRANGEMENT

(71) Applicant: Hahn-Schickard-Gesellschaft für angewandte Forschung e.V., Villingen-Schwenningen (DE)

(72) Inventors: Philipp RAIMANN, Villingen-Schwenningen (DE); Sophie BILLAT, Villingen-Schwenningen (DE); Frank HEDRICH, Villingen-Schwenningen (DE)

(73) Assignee: Hahn-Schickard-Gesellschaft für angewandte Forschung e.V., Villingen-Schwenningen (DE)

(21) Appl. No.: 19/084,791

(22) Filed: Mar. 20, 2025

Related U.S. Application Data
(63) Continuation of application No. PCT/EP2023/075915, filed on Sep. 20, 2023.

(30) Foreign Application Priority Data
Sep. 20, 2022 (DE) 102022209905.2

(51) Int. Cl. G01N 25/20; G01N 25/18; G01N 33/00

(52) U.S. Cl. CPC G01N 25/20; G01N 25/18; G01N 33/0027

(57) ABSTRACT

A sensor arrangement having at least one sensor cell and an evaluation, wherein the at least one sensor cell can be excited thermally by means of a heater; wherein the sensor cell is configured to form an oscillation behavior in dependence on a gas property of a gas surrounding the sensor cell, in particular heat conductivity, volume heat capacity, temperature and/or pressure, and is excited by means of an excitation frequency, and wherein a first excitation frequency or a first evaluation frequency is used for a first measurement, and wherein a second excitation frequency or evaluation frequency is used for a second measurement, wherein the first excitation frequency differs from the second excitation frequency, or wherein the first evaluation frequency differs from the second evaluation frequency; and wherein the evaluation is configured to determine heat conductivity based on the first measurement and volume heat capacity based on the second measurement.

### CROSS-REFERENCE TO RELATED APPLICATIONS

This application is a continuation of copending International Application No. PCT/EP2023/075915, filed Sep. 20, 2023, which is incorporated herein by reference in its entirety, and additionally claims priority from German Application No. 102022209905.2, filed Sep. 20, 2022, which is also incorporated herein by reference in its entirety.

### TECHNICAL FIELD

Embodiments of the present invention relate to a sensor arrangement and to a corresponding evaluation method. Further embodiments relate to a flow sensor having a corresponding sensor arrangement and/or to a pressure sensor having a corresponding sensor arrangement.

### BACKGROUND OF THE INVENTION

The output signals of thermal flow sensors (in general flow sensors) are not only influenced by the flow rate (1/min) but also by gas properties, like density p, heat conductivity k and/or specific heat capacity c of the flowing medium, for example. The gas properties in turn are dependent on temperature and pressure. If, for example, the gas composition, temperature and/or pressure change with a constant flow rate, the output signal will also change, which may erroneously be interpreted as a change in the flow rate. Thus, thermal flow sensors are either calibrated for a gas/gas mixture or the gas properties have to be determined by further additional sensors in order to compensate the resulting output signal using algorithms. Signal compensation is more precise when the sensors are located in direct proximity to the flow sensor.

Consequently, the conventional technology is calibrating flow sensors to gas properties (known pressure, temperature and gas composition) or allowing signal compensation by additional independent MEMS sensors (environmental sensor). In accordance with the conventional technology, sensors having different measuring principles have to be integrated. In typical sensor arrangements of low complexity, heat conductivity and volume heat capacity cannot be determined easily so as to allow calibration. Therefore, there is need for an improved approach.

Embodiments of the present invention are based on the object of providing a concept which allows determining volume heat capacity and heat conductivity in a reliable and precise manner using a measuring arrangement of low complexity.

### SUMMARY

An embodiment may have a sensor arrangement having at least one sensor cell and an evaluation, wherein the at least one sensor cell can be excited thermally by means of a heater; wherein the sensor cell is configured to form an oscillation behavior in dependence on a gas property of a gas surrounding the sensor cell, in particular a heat conductivity and/or volume heat capacity and/or temperature and/or pressure, wherein the sensor cell is excited by means of at least one excitation frequency, and wherein a first excitation frequency or a first evaluation frequency is used for a first measurement, and wherein a second excitation frequency or evaluation frequency is used for a second measurement, wherein the first excitation frequency differs from the second excitation frequency, or wherein the first evaluation frequency differs from the second evaluation frequency; and wherein the evaluation is configured to determine heat conductivity of the surrounding gas based on the first measurement and volume heat capacity of the surrounding gas based on the second measurement, wherein the first excitation frequency is smaller by at least a factor of 2 or at least a factor of 4 than the cutoff frequency of the sensor, and wherein the second excitation frequency is greater by at least a factor of 2 or at least a factor of 4 than the cutoff frequency of the sensor; and/or wherein the first evaluation frequency is smaller by at least a factor of 2 or at least a factor of 4 than the cutoff frequency of the sensor, and wherein the second evaluation frequency is greater by at least a factor of 2 or at least a factor of 4 than the cutoff frequency of the sensor.

Another embodiment may have a flow sensor having an inventive sensor arrangement as mentioned above, wherein the flow sensor is configured to determine a flow while considering the heat conductivity and volume heat capacity determined.

Another embodiment may have a pressure sensor having an inventive sensor arrangement as mentioned above, wherein the pressure sensor is configured to determine the pressure while considering the volume heat capacity and heat conductivity.

According to another embodiment, a method for evaluating an inventive sensor arrangement as mentioned above may have the steps of: exciting the sensor by means of an excitation frequency, wherein a first excitation frequency or a first evaluation frequency is used for a first measurement, and wherein a second excitation frequency or evaluation frequency is used for a second measurement, wherein the first excitation frequency differs from the second excitation frequency, or wherein the first evaluation frequency differs from the second evaluation frequency; and determining heat conductivity based on the first measurement and volume heat capacity based on the second measurement; wherein the first excitation frequency is smaller by at least a factor of 2 or at least a factor of 4 than the cutoff frequency of the sensor, and wherein the second excitation frequency is greater by at least a factor of 2 or at least a factor of 4 than the cutoff frequency of the sensor; and/or wherein the first evaluation frequency is smaller by at least a factor of 2 or at least a factor of 4 than the cutoff frequency of the sensor, and wherein the second evaluation frequency is greater by at least a factor of 2 or at least a factor of 4 than the cutoff frequency of the sensor.

Still another embodiment may have a non-transitory digital storage medium having stored thereon a computer program for performing a method for evaluating an inventive sensor arrangement as mentioned above having the steps of: exciting the sensor by means of an excitation frequency, wherein a first excitation frequency or a first evaluation frequency is used for a first measurement, and wherein a second excitation frequency or evaluation frequency is used for a second measurement, wherein the first excitation frequency differs from the second excitation frequency, or wherein the first evaluation frequency differs from the second evaluation frequency; and determining heat conductivity based on the first measurement and volume heat capacity based on the second measurement; wherein the first excitation frequency is smaller by at least a factor of 2 or at least a factor of 4 than the cutoff frequency of the sensor, and wherein the second excitation frequency is greater by at least a factor of 2 or at least a factor of 4 than the cutoff frequency of the sensor; and/or wherein the first evaluation frequency is smaller by at least a factor of 2 or at least a factor of 4 than the cutoff frequency of the sensor, and wherein the second evaluation frequency is greater by at least a factor of 2 or at least a factor of 4 than the cutoff frequency of the sensor, when the computer program is run by a computer or the evaluation.

Embodiments of the present invention provide a sensor arrangement comprising at least one sensor cell, and an evaluation. The at least one sensor cell can be excited thermally by means of a heater or can be excited to oscillate thermally. The sensor cell is configured to form a corresponding (thermal) oscillation behavior in dependence on a gas property of a gas surrounding the sensor cell, in particular heat conductivity and/or volume heat capacity and/or temperature and/or pressure. In accordance with embodiments, the heater, which is exemplarily implemented as a self-supporting bridge structure, oscillates. The sensor cell is excited by means of an excitation frequency, wherein a first excitation frequency or a first evaluation frequency is used for a first measurement, and wherein a second excitation frequency or second evaluation frequency is used for a second measurement. The first excitation frequency differs from the second excitation frequency and the first evaluation frequency differs from the second evaluation frequency. The evaluation is configured to determine heat conductivity based on the first measurement and volume heat capacity based on the second measurement. It is to be pointed out here that, in accordance with embodiments, the first excitation frequency and/or the second excitation frequency may be greater than or equaling 0 Hz. This also means that, for a first measurement, excitation (or excitation for a second measurement) may take place at 0 Hz, which means that excitation may, for example, be done using a DC current. For example, the first excitation frequency equals 0, wherein the excitation energy for the first measurement is greater than 0. In the second measurement, a second excitation frequency of greater than 0 Hz is used, for example. In accordance with embodiments, the second excitation frequency may, of course, equal 0 Hz, that is using an excitation energy for the second measurement of greater than 0, wherein in this case the first measurement using an excitation frequency of greater than 0 is used.

Embodiments of the present invention are based on the finding that, in different excitations, for example using different excitation frequencies, differentiated sensitivities to different physical parameters can be implemented in association to different physical groups, i. e. for group 1 (including heat conductivity) and for group 2 (including volume heat capacity). Due to the different excitations/different excitation frequency and also due to different evaluation frequencies, differently high sensitivities to the different physical parameters are formed, which allows determining volume heat capacity cv and heat conductivity k independently. Here, the (single) sensor is operated in a first frequency range which exhibits high a sensitivity to heat conductivity k and at the same time low cross sensitivity to volume heat capacity cv. For a second measurement, the (same) sensor is operated in a frequency range which exhibits high sensitivity to volume heat capacity cv and at the same time low cross sensitivity to heat conductivity k.

In other words, this means that, in accordance with embodiments, two measurements are performed using different excitations. Measurement 1 can be performed using a first excitation, which, for example, comprises a first excitation frequency, whereas measurement 2 is performed using a different excitation, for example, a greater or smaller, i. e. different, excitation frequency. Alternatively, it would also be conceivable for excitation 1 to comprise a DC excitation (excitation frequency equaling 0), whereas measurement 2 is performed using a different excitation with an excitation frequency of greater than 0. A further alternative would be evaluating a sensor at different evaluation frequencies.

In accordance with embodiments, the first excitation frequency differs from the second excitation frequency by at least a factor of 2 or at least a factor of 4 or even at least a factor of 10. In analogy, the first and second evaluation frequencies may differ by at least a factor of 2, at least a factor of 4 or at least a factor of 8. Here, fixed first excitation frequencies and second excitation frequencies or fixed evaluation frequencies and second evaluation frequencies can be used.

In accordance with an embodiment, the excitation frequency is determined as a function of the cutoff frequency of the sensor. For example, the first excitation frequency may be smaller by at least a factor of 2 or at least a factor of 4 than the cutoff frequency of the sensor, wherein the second excitation frequency is greater by at least a factor of 2 or at least a factor of 4 than the cutoff frequency of the sensor. In accordance with an alternative/additive embodiment, the evaluation frequency may be smaller by at least a factor of 2 or at least a factor of 4 than the cutoff frequency of the sensor, whereas the second evaluation frequency is greater by at least a factor of 2 or at least a factor of 4 than the cutoff frequency of the sensor.

The result, in accordance with embodiments, is a difference in sensitivity in the two measurements. For example, the sensitivity to volume heat capacity in the second measurement may be higher by at least a factor of 3 or at least a factor of 4 or at least a factor of 5 than the sensitivity to volume heat capacity in the first measurement. The sensitivity to heat conductivity in the second measurement may be higher by at least a factor of 1.1 or at least a factor of 1.2 than the sensitivities to heat conductivity in the second measurement.

In accordance with embodiments, the sensor cell is excited by means of an excitation frequency in the first and second measurements or, in the special case described above, in at least one of the two measurements. The periodic excitation may, for example, take place by means of a square wave voltage. In accordance with further embodiments, it would also be conceivable for the excitation frequency to be configured to be varying, for example as a Chirp signal or as a Dirac signal. Here, for evaluation, different evaluation frequencies are selected for the first and second measurements.

It is common to both variations discussed above that, in accordance with further embodiments, the measurements may take place at different points in time (measurement 1 point in time t1, measurement at point in time t2).

Sensor: In accordance with embodiments, the sensor cell may comprise a cavity having a heater, or a heat sink having a spaced-apart heater (or a heating rib spaced apart from the heat sink). The heater or heating rib may be configured to oscillate thermally and thus form the (thermal) oscillation behavior. In accordance with embodiments, it would also be conceivable for the heater to be formed by the heating rib, like in the form of a self-supporting structure or self-supporting bridge structure.

In accordance with further embodiments, the sensor cell comprises a detector configured to detect the oscillation behavior. The detector can be arranged separately from the heater. In accordance with further embodiments, the detector may also be integrated in the heater as follows. The heater is excited to oscillate (thermally) wherein then a resistive evaluation of the temperature signal may take place in the same heater. In accordance with embodiments, a current flows through the heater, which is, for example, made from metal or another conductive material.

In accordance with embodiments, the evaluation is configured to determine the oscillation behavior of the sensor using the dynamic temperature response and/or using the amplitude and/or using the frequency and/or using the phase. In accordance with embodiments, the evaluation may be implemented as an ASIC. Here, the ASIC may be integrated in a chip or monolithically in the chip which also accommodates the sensor cell.

Further embodiments provide a flow sensor comprising a corresponding sensor arrangement. The flow sensor is configured to determine a flow (volume flow or gas flow) while considering the heat conductivity and volume heat capacity determined. It is of advantage here that determining takes place in a compensated manner.

Further embodiments provide a pressure sensor configured to determine the pressure while considering the volume heat capacity and/or heat conductivity.

In the two applications of the pressure sensor and flow sensor just mentioned, it is of advantage that, by determining heat conductivity and heat capacity, the gas or gas mixture may be unknown so that nevertheless the correct volume flow or correct pressure can be determined.

A further embodiment provides a method comprising the steps of:
exciting the sensor (10) by means of an excitation frequency, wherein a first excitation frequency or a first evaluation frequency is used for a first measurement (M1), and wherein a second excitation frequency or evaluation frequency is used for a second measurement (M2), wherein the first excitation frequency differs from the second excitation frequency, or wherein the first evaluation frequency differs from the second evaluation frequency; and
determining heat conductivity (k) based on the first measurement (M1) and volume heat capacity (cv) based on the second measurement (M2).

In accordance with further embodiments, the method may be computer-implemented.

### BRIEF DESCRIPTION OF THE DRAWINGS

Before discussing embodiments of the present invention below referring to the appended drawings, it is pointed out that elements and structures of equal effect are provided with equal reference numerals so that the description thereof is mutually applicable or interchangeable.

FIGS. 1a and 1b schematically show, in a sectional view (FIG. 1a), top view (FIG. 1b), a sensor cell for being used in embodiments;

FIGS. 2a-2q show schematic illustrations of sensor cells for being used in extended embodiments;

FIGS. 3a and 3b show a schematic embodiment of a sensor arrangement comprising two sensor cells in accordance with a comparison aspect;

FIGS. 3c and 3d show schematic diagrams for illustrating potential sensor cells dimensions in accordance with embodiments;

FIGS. 4a and 4b show schematic diagrams for discussing the sensitivity to heat conductivity and volume heat capacity for two sensor cells in accordance with comparison aspects;

FIG. 5 shows a schematic block circuit diagram of sensor cell comprising evaluation electronics in accordance with a main embodiment;

FIGS. 6a and 6b show schematic diagrams for discussing the sensitivity to heat conductivity and volume heat capacity for two sensor cells in accordance with embodiments;

FIGS. 7a and 7b show schematic diagrams for illustrating sensitivities plotted over frequency for discussing potential configurations in accordance with embodiments;

FIG. 8 shows a schematic diagram for illustrating the dependence between sensor sensitivity and measuring gas;

FIGS. 9a, 9b, 9c and 9d show schematic illustrations of potential applications in accordance with embodiments; and

FIGS. 10a-10c show illustrations for discussing evaluation in accordance with an extended embodiment.

### DETAILED DESCRIPTION OF THE INVENTION

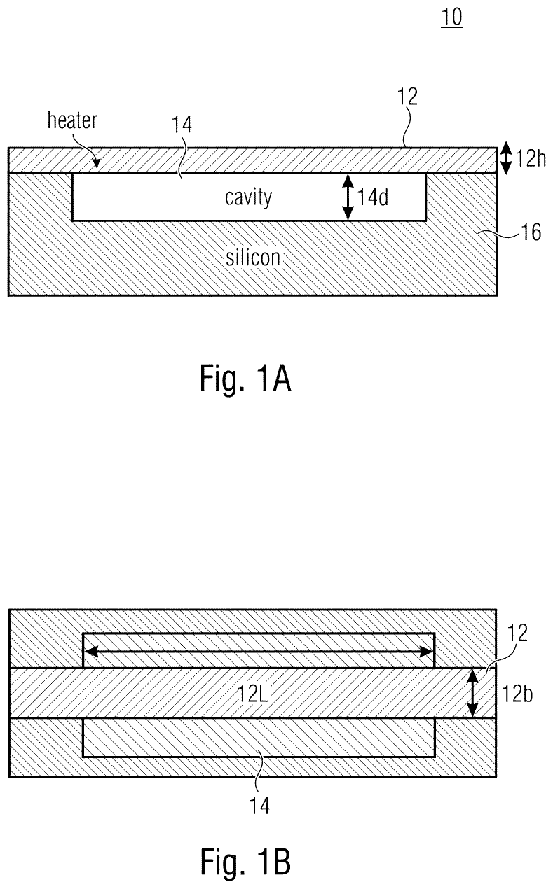

FIG. 1a shows a sectional view of a sensor cell 10 and FIG. 1b a top view. The sensor cell 10 comprises a heater 12 which is arranged, for example, as a self-supporting structure above a cavity 14. The cavity 14 may, for example, be embedded in a silicon substrate 16. The heater 12 can be excited to oscillate thermally by excitation by means of a drive frequency. Here, an alternating voltage or current having the corresponding frequency flows through the same, for example. Starting from the excitation, the result is a frequency-dependent temperature superelevation or, if applicable, low-pass behavior. This temperature superelevation or low-pass behavior is dependent on geometry parameters and material properties. Essential geometry parameters are, for example, the height of the heater 12h, the width of the heater 12b, and the length of the heater 12l. A further parameter is the volume of the cavity 14 which basically is dependent on the height of the cavity 14d. The silicon substrate 16 or the lower side of the cavity 14 serves as a temperature sink for the heater 12, wherein heat transfer in the temperature sink depends on the thickness 14d of the cavity 14.

A sensor cell 10 excited to oscillate in this way is configured to oscillate at a corresponding frequency. This frequency is dependent on physical parameters of the surrounding gas, both on the side of the cavity 14 and on the side opposite the cavity 14, which serves as the measuring side, for example. Influential factors are temperature, pressure, but in particular heat conductivity and volume heat capacity, for example. Conversely, this allows these physical parameters of heat conductivity and/or volume heat capacity to be determined starting from the oscillation behavior of the heater. Here, the oscillation behavior is monitored by means of a detector (not illustrated), for example.

The determination method is as follows:
Heater is excited periodically (current or voltage) and heats up (Joule heating).
Temperature of the heater varies and is dependent on the heat exchange with the surrounding gas (the gas to be analyzed/surrounding the self-supporting heat structure 12).
Heat conductivity and volume heat capacity influence dynamic heat dissipation to the gas.
As a consequence, the dynamic temperature response of the heater 12 (amplitude and phase, for example) can be measured and resistive or thermos-electric monitoring of the thermal response may take place for detection. The following heat transfer temperature T results, for example. T=function (L, h, b, d, kₕ, cvₕ, k_{gas}, cv_{gas}) with the following surrogate parameters: Rₕₑₐₜₑᵣ=L/(h*b*kₕ); Cₕₑₐₜₑᵣ=cvₕ*b*h*L; R_{gas}=d/(L*b*k_{gas}); C_{gas}=d*b*L*cv_{gas}.

If gasses exhibit different k_{gas} and cv_{gas}, the amplitude and the dynamic behavior differ with frequency. The system exhibits a high-pass behavior, that is the temperature decreases with an increasing frequency and phase shift increases with an increasing frequency. This frequency-dependent behavior may, apart from sensor dimensioning, also be dependent on the gas properties. If an equilibrium between the scaling factors for width of heater b and width of effective heat transfer area to the gas b_{gas} is assumed, the cutoff frequency is described by the following relation. The scaling factors can be eliminated for b_{gas}=b and the following applies:

$${f}_{cutoff} = \frac{h \cdot {k}_{h}}{2 \cdot π \cdot {L}^{2} ( {cv}_{h} \cdot h + {cv}_{gas} \cdot d )} + \frac{{k}_{gas}}{2 \cdot π \cdot d ( {cv}_{h} \cdot h + {cv}_{gas} \cdot d )}$$

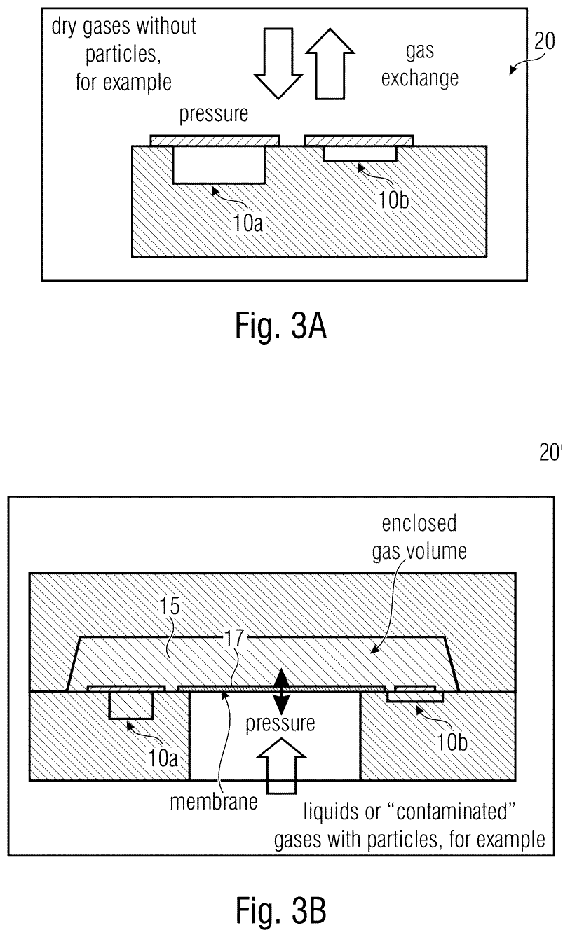

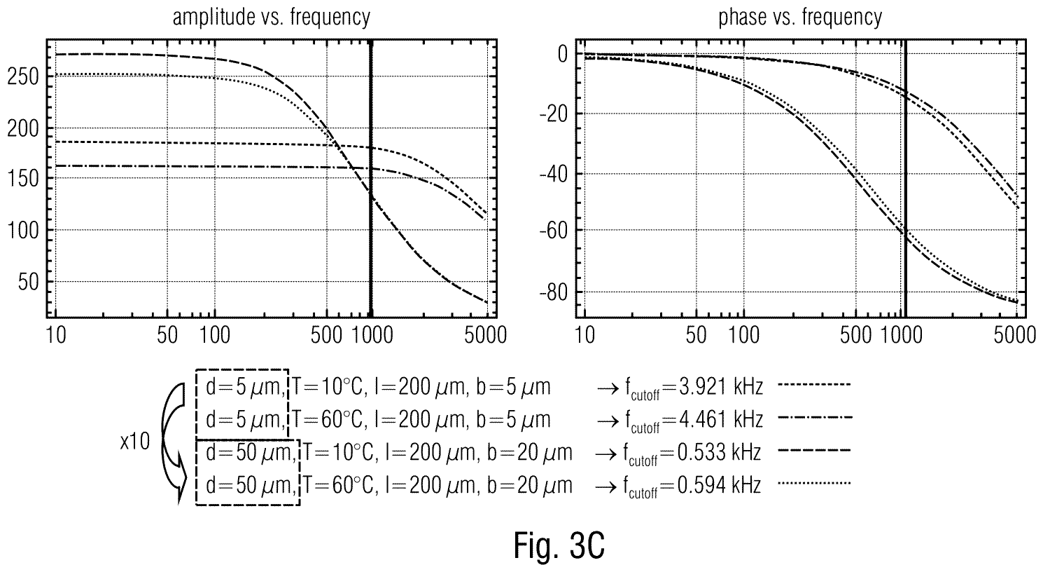

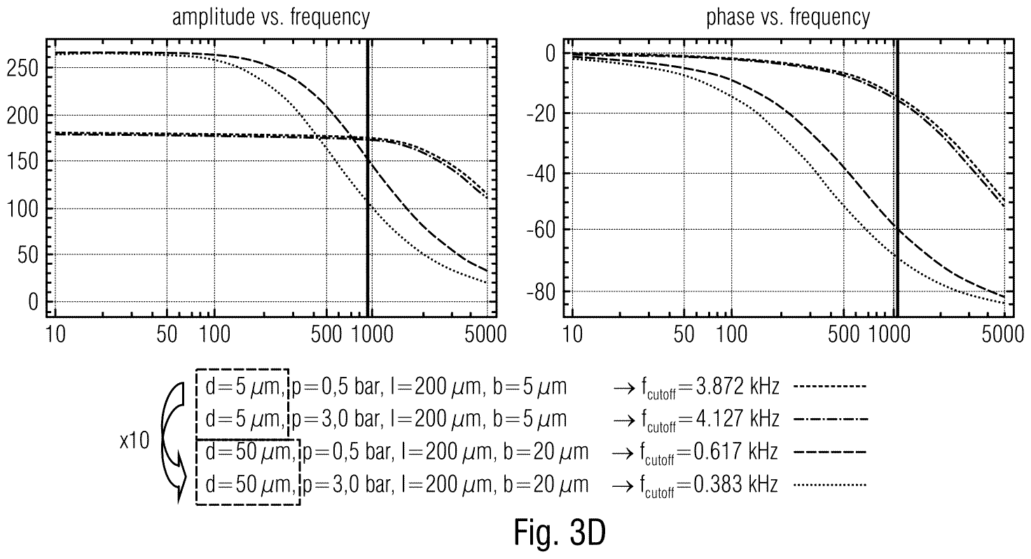

It can be derived from this that the cutoff frequency will be the smaller, the lower heat conductivity and the higher volume heat capacity. This means that an increase in pressure decreases the cutoff frequency of the system. An increase in temperature increases the cutoff frequency of the system. This background, from a physical point of view, results in the finding of the invention that, by using two sensor cells of different dimensioning (maybe with different drive frequency), as are shown, for example, in FIG. 3a or 3b, the heat conductivity k_{gas} or Sₖ and the volume heat capacity S_{cv} or cv_{gas} can be determined independently of each other. Here, a sensor having a high sensitivity to heat conductivity is used in combination with a sensor of high sensitivity to volume heat capacity, for example.

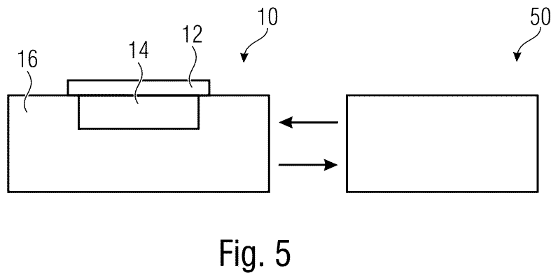

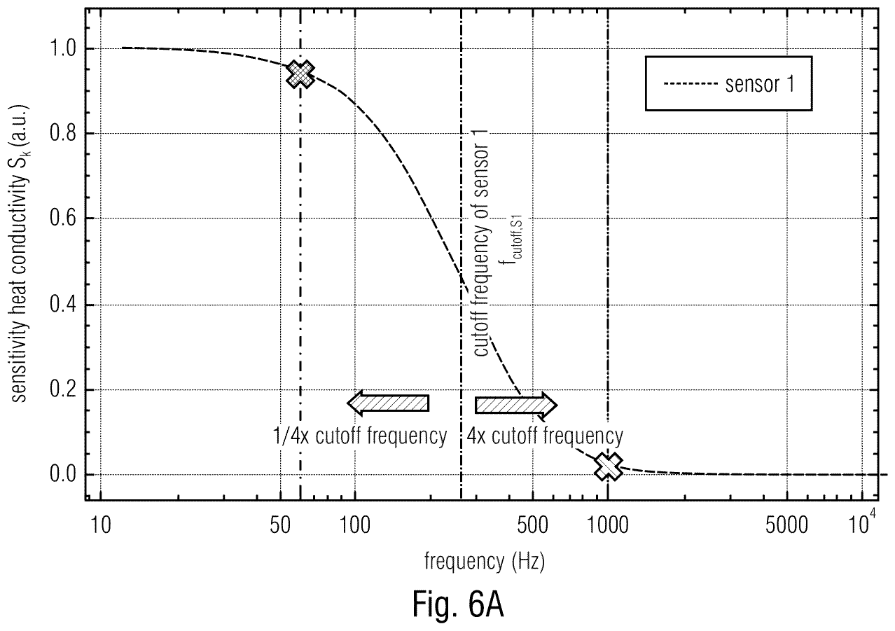

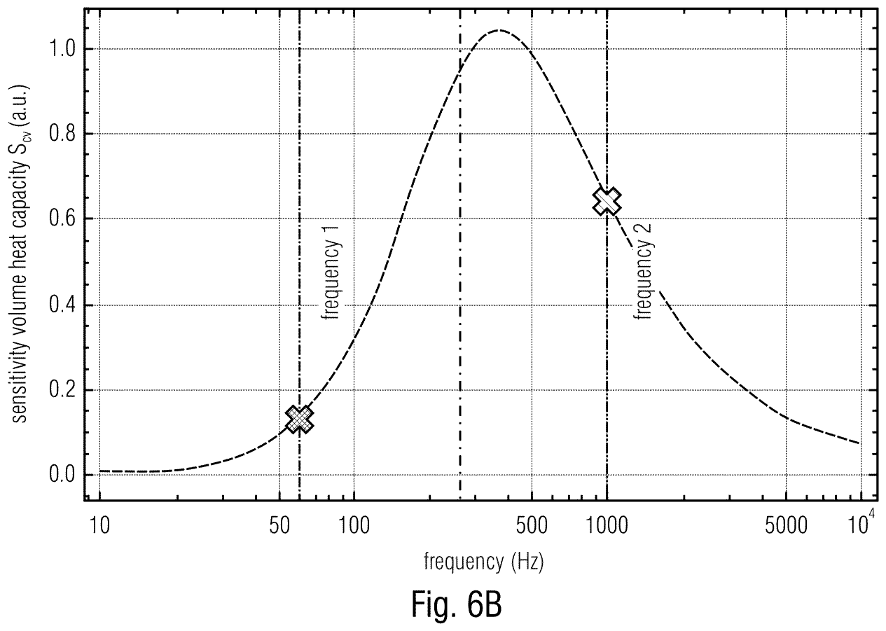

In accordance with embodiments, the sensor includes at least one separate heat element with a surrounding gas volume which is heated periodically and the temperature response of which is determined. In accordance with embodiments, sensors are either read out equally or independently of one another by means of temperature-dependent resistors and/or thermal elements:
Variation 1, see FIGS. 5 and 6: At least one sensor is scanned at two fixed frequencies or over two frequency ranges where the thermal sensor has a sufficiently high difference in the sensitivity to heat conductivity and volume heat capacity
Variation 2, see FIG. 3: Two or more sensors (sensor arrays) are configured by geometrical parameter variation (length, width, layer thickness, shape, height of the cavity) and/or different material properties such that the thermal coupling to the heat sink differs and combines so that it varies sufficiently in the sensitivity to heat conductivity and volume heat capacity when operated in one or more selected frequency ranges or fixed frequencies.

FIG. 3a shows a sensor arrangement 20 comprising a first sensor 10a and a second sensor 10b as a comparison example. As can be recognized, the sensors are dimensioned to be of different sizes, wherein the basic principle corresponding to that from FIG. 1a and FIG. 1b. Both sensor cells 10a and 10b are facing a gas to be examined with their side facing away from the cavity or are embedded in a device such that gas exchange, for example, with dry gasses without particles, may take place.

Potential variation parameters for the different dimensioning of the (two) sensor cells are, for example:
Geometrical parameters (length, width, layer thickness)
A heater may be formed from several heaters (two heaters in parallel, combination of several types of heaters, for example)
Types of heaters (holes, for example, honey comb structure, with membrane, meandering, . . . )
Materials/material combinations (thermal characteristics, passivation, . . . )

FIG. 3 shows a slightly varied setup in which the sensors 10a and 10b in the sensor arrangement 20′ are coupled indirectly via an enclosed volume. The enclosed volume is provided with the reference numeral 15 and encapsulated relative to the environment by a membrane 17. The gas to be measured or the medium to be measured, liquid or contaminated gas with particles acts on the membrane 17.

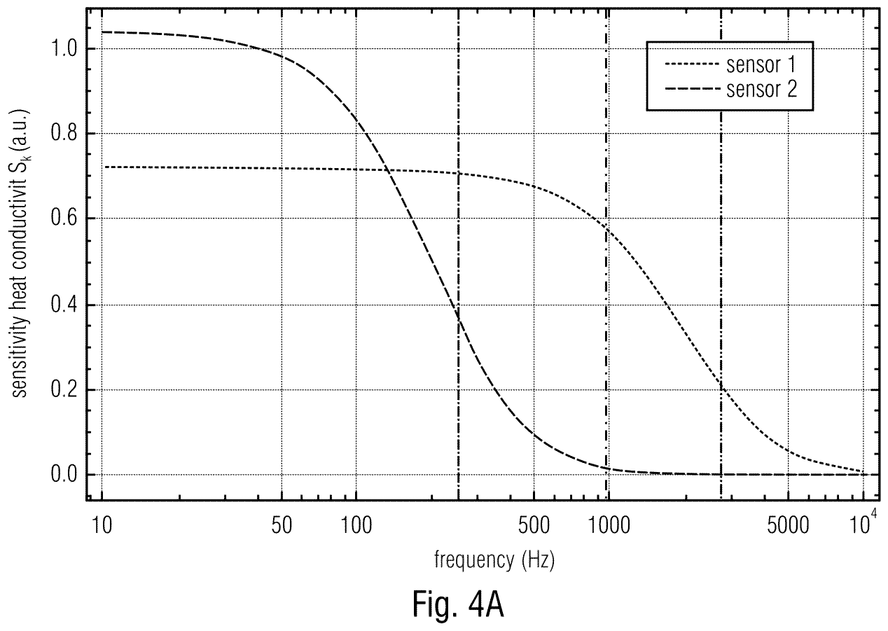

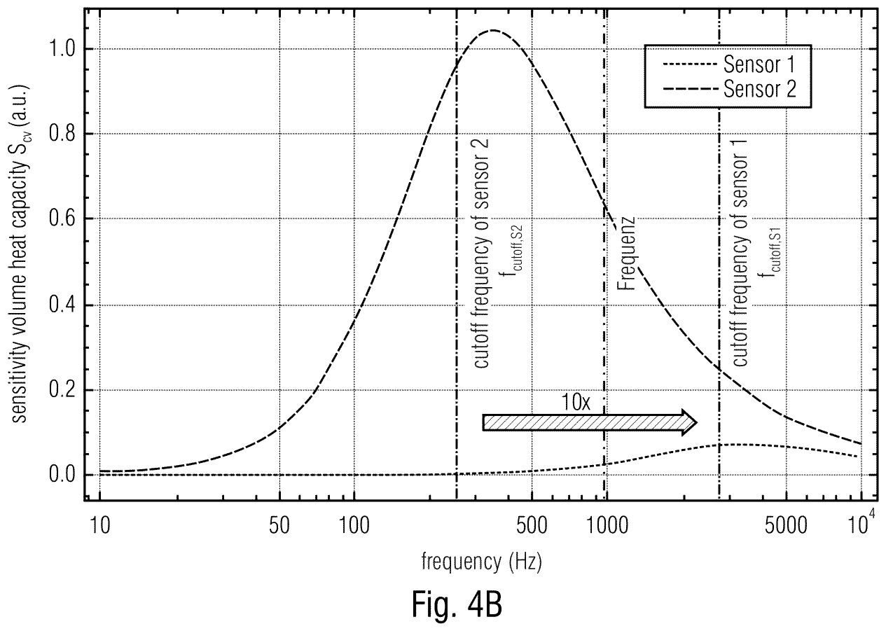

As can be recognized clearly, both in the implementation of FIG. 3a and the implementation of FIG. 3b, the dimensioning of the sensor cells 10a and 10b is different. This means that different sensitivities relative to the quantities to be measured of volume heat capacity cv and heat conductivity k result. This principle can be seen clearly when looking at FIGS. 4a and 4b, for example.

FIG. 4a shows the sensitivity to heat conductivity Sₖ plotted over frequency for two different sensors, whereas FIG. 4b shows the sensitivity to volume heat capacity S_{cv}, again plotted over frequency, for the same two sensors. It can be seen that sensor 2, in particular in a frequency range of 10-100 Hz, is more sensitive than sensor 1, wherein sensor 1 exhibits an improved sensitivity over the entire frequency range or at least up to 1000 Hz. The difference between the sensitivities between sensor 1 and sensor 2 is not significant in these examples so that the operating point of sensor 1 and sensor 2 is important here. If, for example, the operating point is selected to be 350 Hz, sensor 1 is to of advantage as to sensitivity of heat conductivity. It is to be pointed out here that the discussion of the operating points is only exemplarily and varies from one sensor to the next. In the diagram of FIG. 4b, the sensitivity to volume heat capacity S_{cv} is plotted for the two sensors 1 and 2. As can be seen, a significantly higher sensitivity is present in sensor 2 when compared to sensor 1. Here, the operating point would be selected in the range between 350 and 1000 Hz and sensor 2 be operated at the cutoff frequency f_{cutoff,S2}. In this range, sensor 1 advantageously exhibits good sensitivity to heat conductivity so that, when operating the two sensors with equal excitation or evaluation frequency, volume heat capacity and heat conductivity can be determined independently of each other using the two different sensors.

In accordance with a further comparison example, the operating point may vary per sensor. Preferably, sensor 2, for determining heat conductivity, would be operated below its cutoff frequency f_{cutoff,S2}, for example, at ⅓ thereof, to determine heat conductivity. Sensor 2 would be operated in the range of the cutoff frequency or a little higher than cutoff frequency f_{cutoff,S2}, in order to determine the volume heat capacity.

The result are different operating modes corresponding to different comparison examples:
Different dimensioning+different frequencies
Different dimensioning+equal frequencies

Evaluation: When talking about frequency in the embodiments, excitation frequency or evaluation frequency can be assumed. For example, the sensor can be excited at a certain frequency and be evaluated at another one. This is of advantage when using a Chirp signal or a Dirac signal, for example, and different frequencies are gone through. Alternatively, fixed excitation frequencies could be used for both sensors or for the respective sensors 10a and 10b or for two measurements.

It is common to the above comparison examples that two thermal sensors or at least two thermal sensors are excited and read out independently of each other so that the different sensitivities of the two sensors which are either operated differently or are implemented differently, can be made use of. The sensitivities can be calculated as follows:

$${S}_{k} = \frac{\partial T ( {k}_{gas} , {cv}_{gas} , b , L , h , d , {k}_{h} , {cv}_{h} )}{\partial {k}_{gas}} \\ {S}_{cv} = \frac{\partial T ( {k}_{gas} , {cv}_{gas} , b , L , h , d , {k}_{h} , {cv}_{h} )}{\partial {c}_{vgas}}$$

As has been mentioned above, the sensitivities can be adjusted via the operating point.

It is common to all the comparison examples mentioned above that at least two sensors/sensor cells with different dimensioning, for example differing by at least one order of magnitude, are combined. These may, for example, be integrated on a silicon chip (that is monolithically) and thus form a different dynamic behavior with the propagation of heat in gases. Different operating points, in analogy to different dimensioning of the sensors, provide the basis for allowing that gas properties can be determined by determining the amplitude and/or the phase position of the heater with dynamic excitation.

Typical dimensions of sensor cells will be indicated below for an exemplary example. Here, all the dimensions may occur in combination or individually:
Length of the heater: 10-1000 μm
Width of the heater: 1-200 μm
Width of heat transfer: 1-500 μm
Height of the heater: 0.1-2 μm
Height of the cavity: 0.05-500 μm

With these dimensions, sensor cells of different dimensioning and, consequently, different oscillation behavior can be manufactured. The oscillation behavior is expressed in particular using the cutoff frequency f_{cutoff}. In FIG. 3c, four different sensors with different cutoff frequencies and thus with different oscillation behaviour are listed and illustrated in corresponding diagrams (amplitude vs. phase and phase vs. frequency). As can be recognized, by varying the cavity height d, a significant shift in the cutoff frequency by a factor of, for example, 10 can be achieved. This sensor is configured for a constant pressure of, for example, 1 bar and varying temperatures in a range from 10 to 60° C. Even with a constant temperature (like 24° C., for example) and varying pressure in the range from 0.5 to 3.0 bar, the result is similar, as can be recognized in FIG. 3d. Here, too, four sensors are illustrated, wherein it can again be recognized that the cavity height (in general height of the heating element above the heat sink) has a significant influence on the cutoff frequency f_{cutoff}.

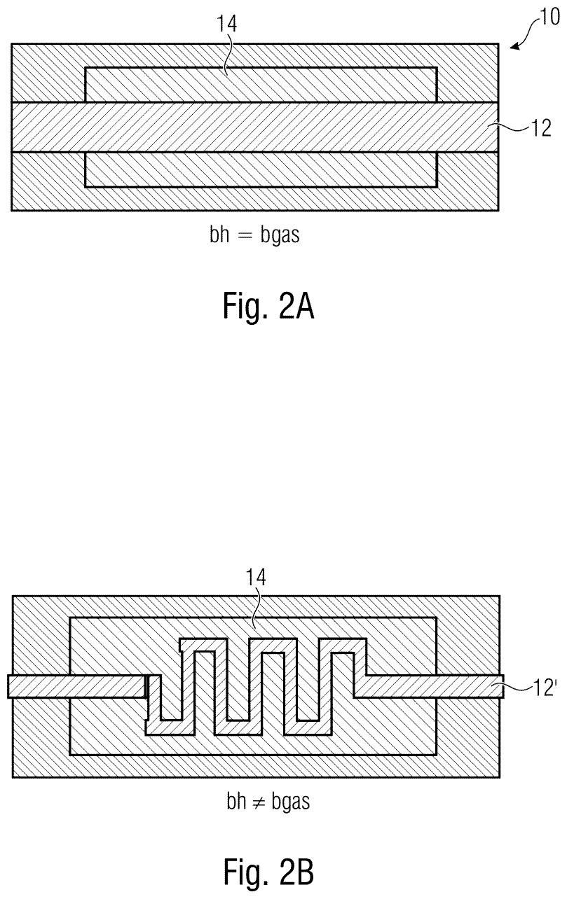

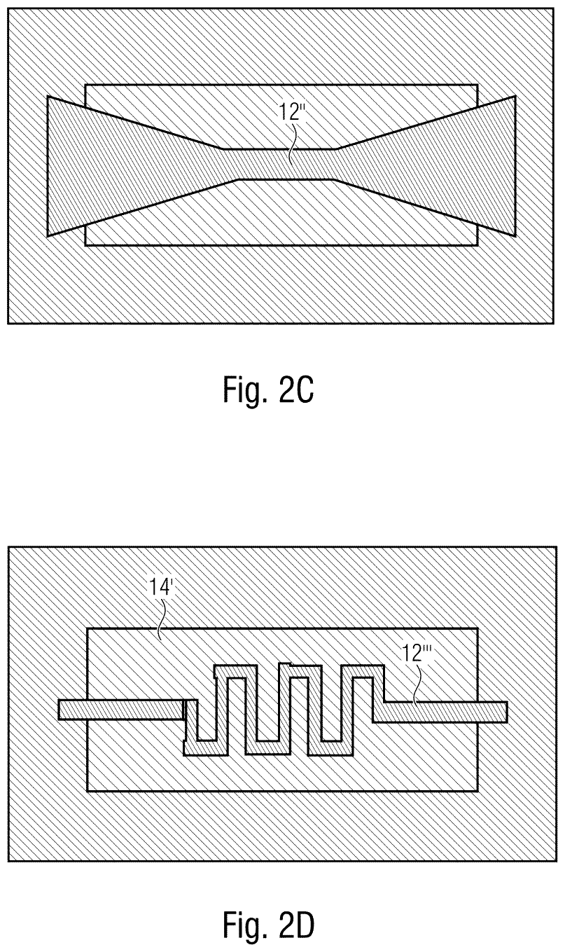

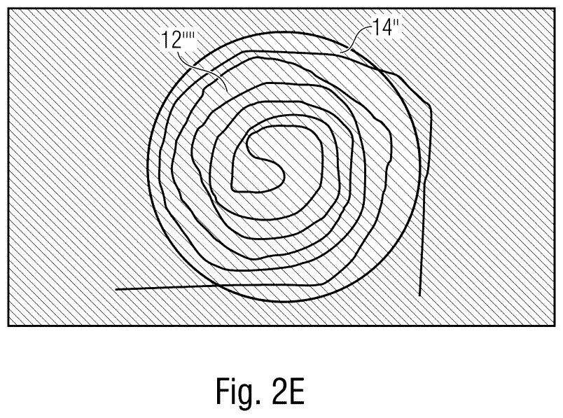

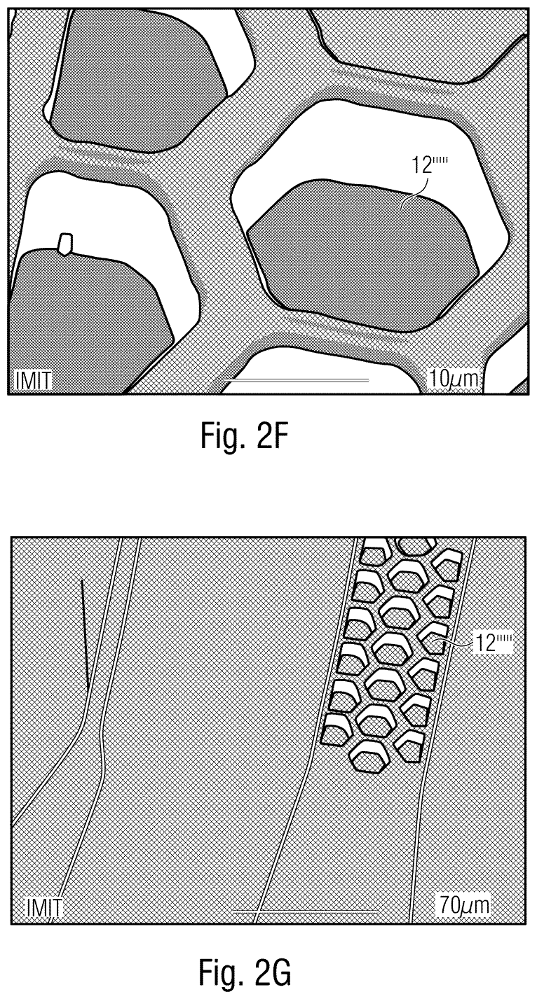

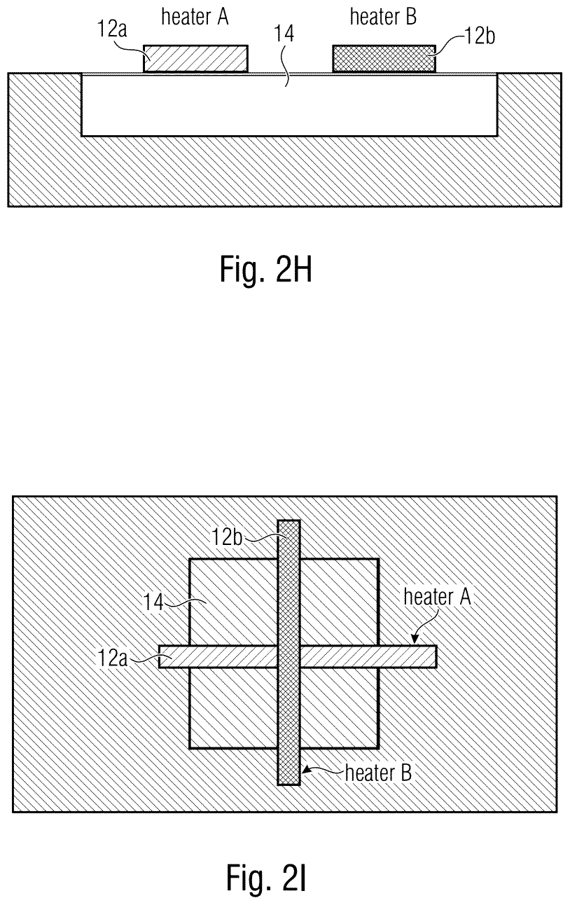

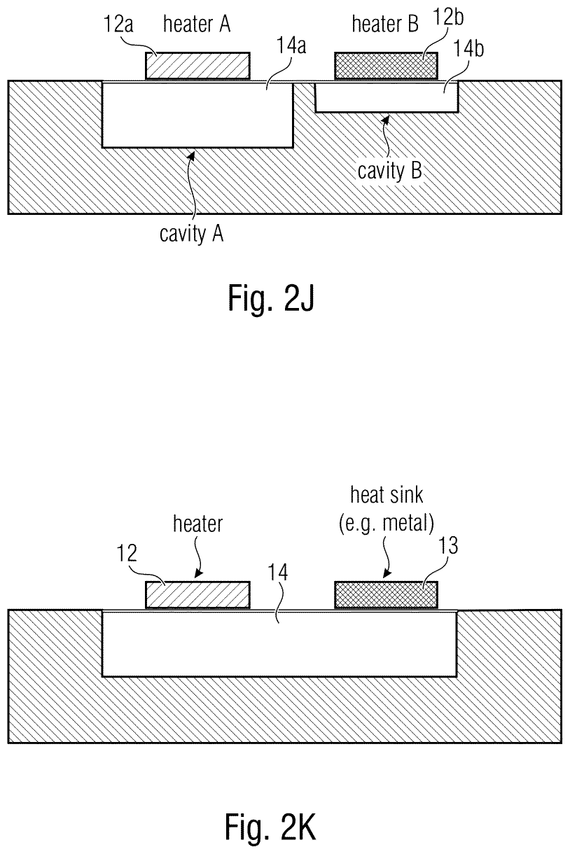

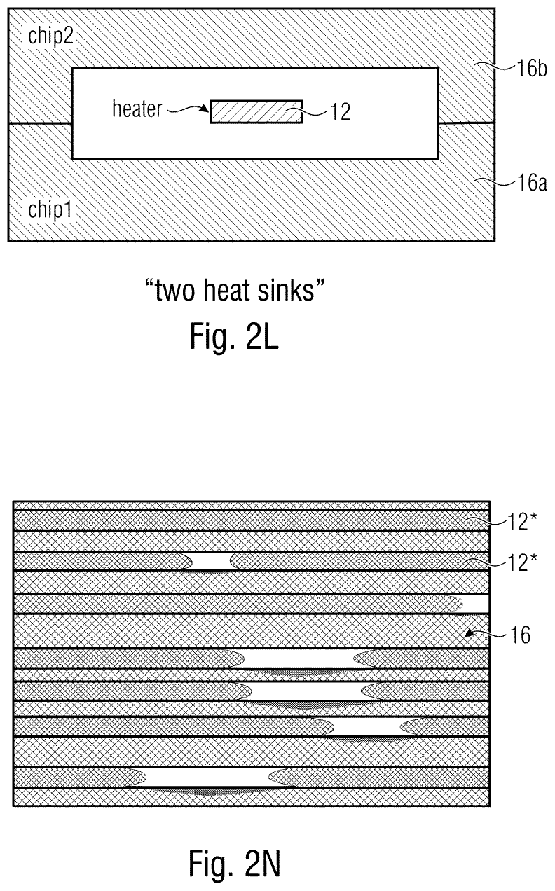

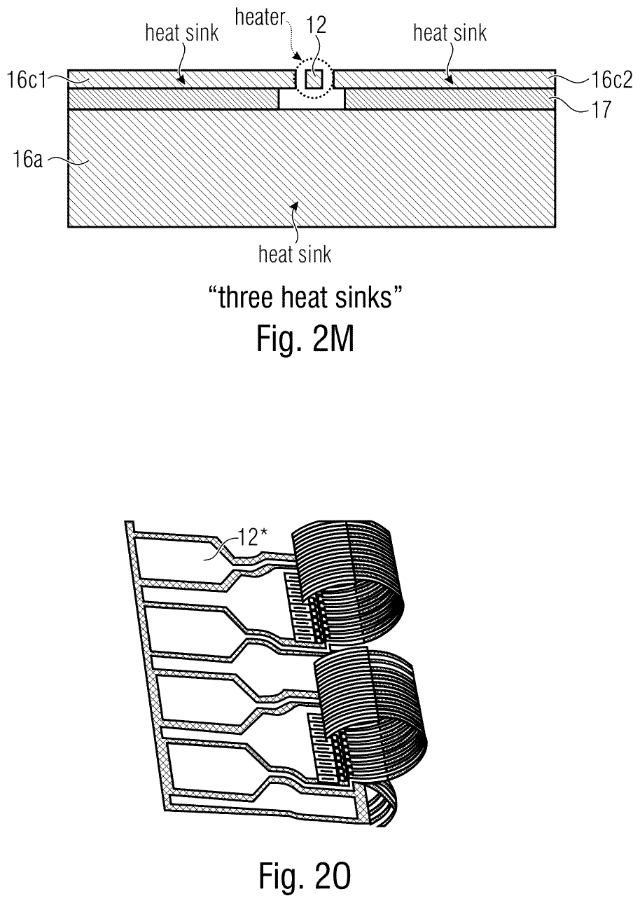

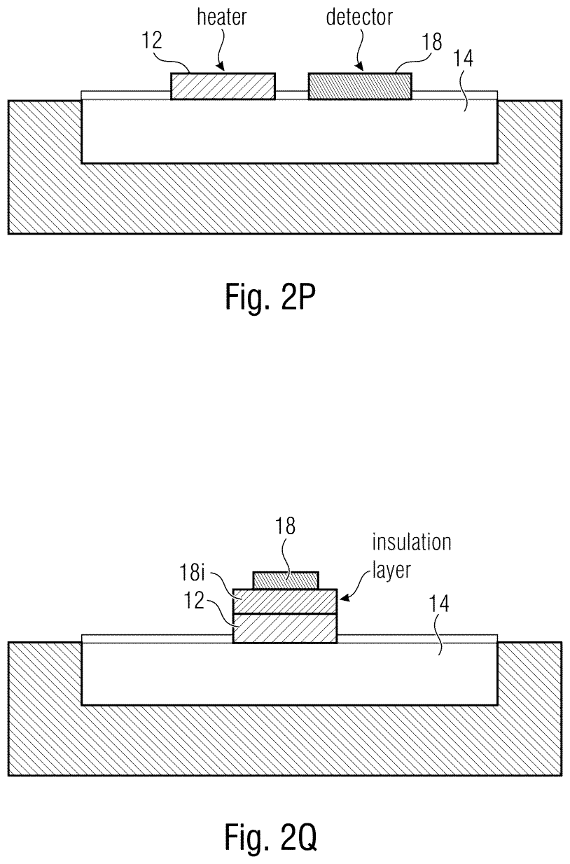

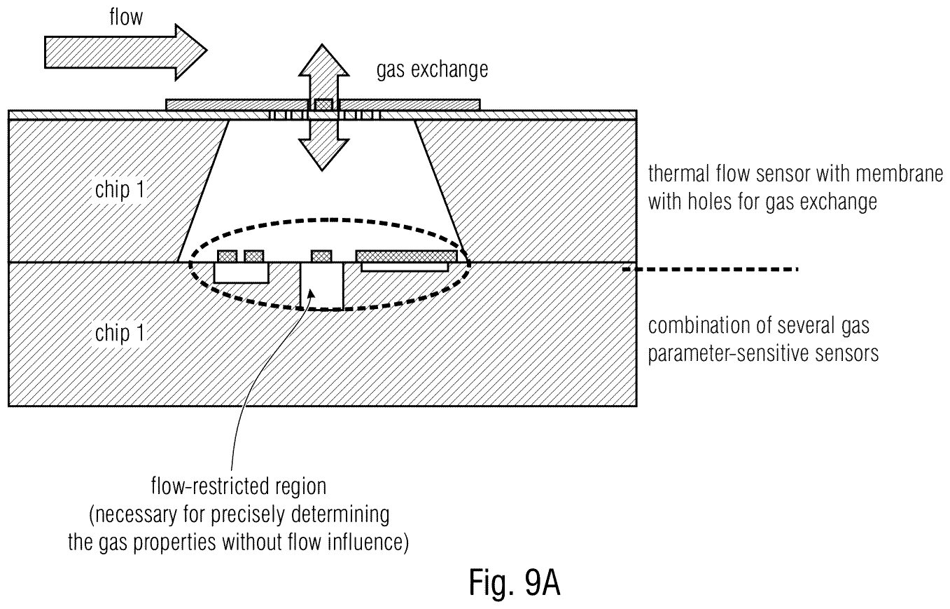

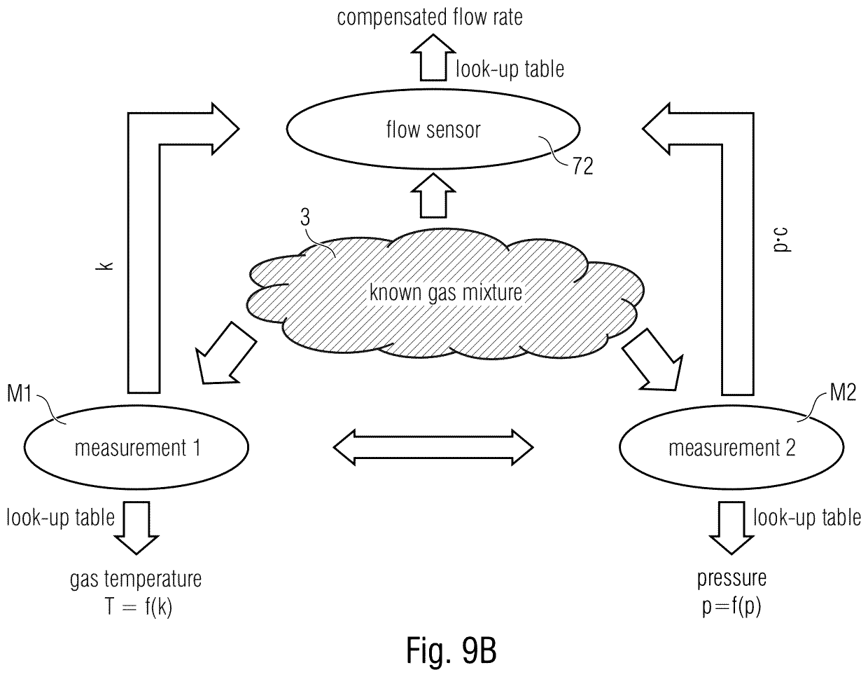

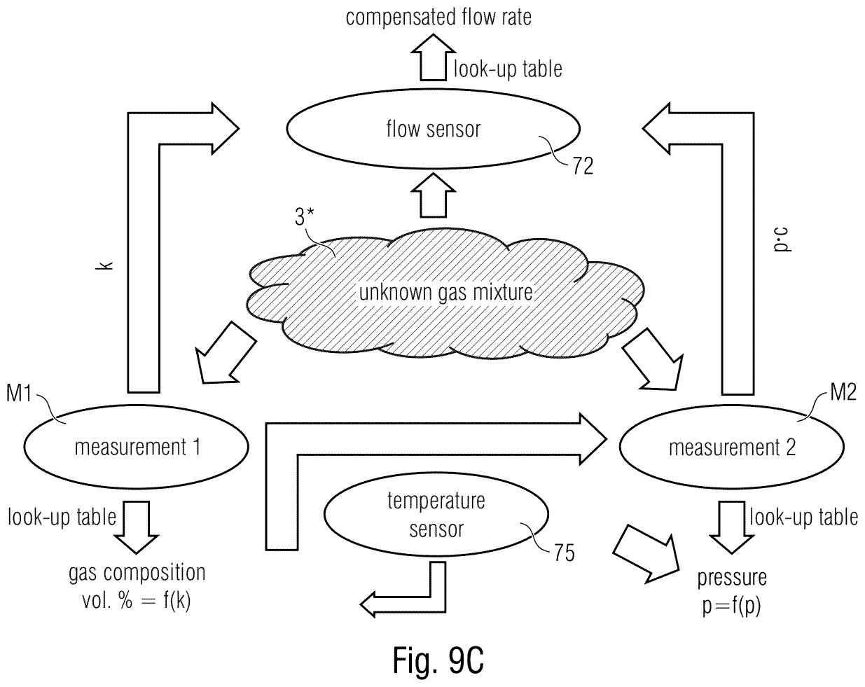

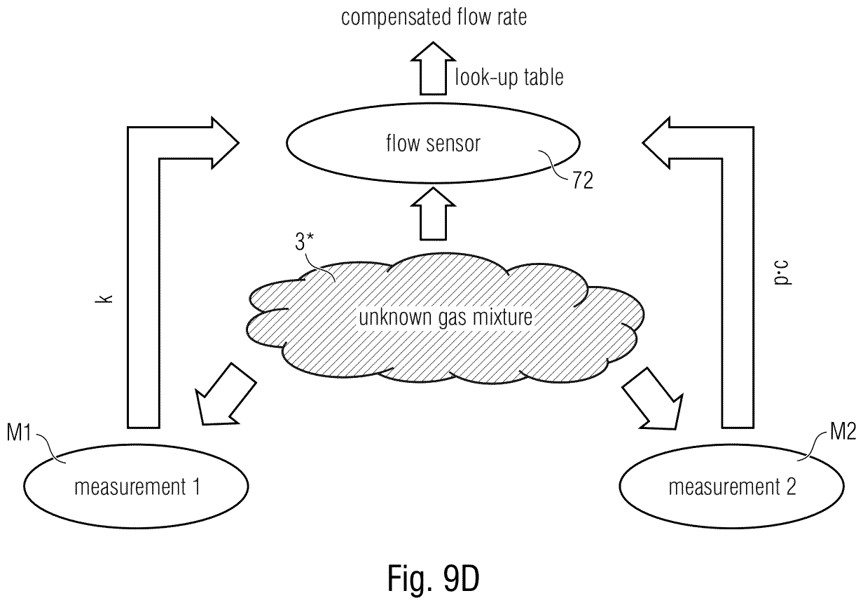

Excitation: In the above comparison examples, it has been assumed, for example, that the heater is excited periodically by means of a square wave signal and a sine signal, wherein the responsiveness of the heater, that is the oscillation behaviour or thermal oscillation behaviour of the heater, can be monitored by only a few thermal elements or changes in resistance. The result of modelling, with an excitation of, for example, 1 kHz, is: amplitude (and phase) of the heater exhibits a dependence on the gas pressure with a (large) gas volume (d=50 μm, b=20 μm) and is insensitive to changes in temperature. The amplitude of the heater exhibits a dependence on the gas temperature with a (small) gas volume (d=5 μm, b=5 μm) but is insensitive to changes in pressure. In this example, too, it has shown that a combination of two or more sensor cells with different dimensioning (d, b, L), advantageously on a chip, is of advantage and that, apart from determining the volume heat capacity and heat conductivity, it also allows providing wide-range sensors for different measuring quantities of temperature and pressure. In accordance with comparison examples, a gas-independent wide-range pressure sensor (a few mbar to a few bar) can be provided without any mechanical components (diaphragm). Of course, using this comparison example, gas properties can also be determined (determining heat conductivity and volume heat capacity). In the next step, this allows determining the so-called temperature conductivity or product from density and heat conductivity. Remark: the temperature conductivity is defined as heat conductivity/(density*specific heat capacity), i.e. a=k/(ρ*c). These quantities can advantageously be used for precise on-chip signal compensation in flow sensors or pressure sensors, as will be discussed below referring to FIG. 9. It is also to be pointed out here that, for providing differently dimensioned sensors, the geometry of the heater may also be varied. Meandering shapes, honeycomb-shaped heating structures, heat mirrors, as discussed in connection with FIG. 2, are conceivable.

In accordance with embodiments, an arrangement of the sensors of FIG. 3 or FIG. 5 with micro-technological manufacturing methods can be produced to be process-compatible to further (thermal) sensors and thus offers a high integration density for multi-parameter applications (like gas composition and flow rate, for example). Due to the small dead volume, the arrangement may additionally be operated to be highly dynamic. In accordance with embodiments, isolated heating structures surrounded by gas are realized by sacrificial layer technology (surface micromechanics) or bulk micromechanics (dry etching, . . . ). The heating elements provided in this way can be heated periodically by Joule heating. At the same time, the temperature response of the heater is monitored. This arrangement allows strongly miniaturizing the sensor structure. The properties of the gas influence the resulting temperature response of the heating element (amplitude, phase shift). Due to the small space requirements, several of these sensors can easily be integrated with thermal flow sensors on the wafer level. Additionally, only thermal conversion principles are employed. This combination renders the system unique.

In accordance with comparison examples, the sensors are sufficiently insensitive and can be operated in the same frequency range, for example if the resulting cutoff frequency differs at least by the factor of 10 due to geometrical parameter variation (length, width, layer thickness, shape, height of the cavity) and/or different material properties.

Examples of decreasing the cutoff frequency
increasing the length of the heater
increasing the height of the cavity.

In accordance with embodiments, high sensitivity to heat conductivity is obtained with excitations smaller than the cutoff frequency, for high sensitivity to volume heat capacity, excitations above the cutoff frequency are of advantage.
Gas properties can be derived using the signal amplitude and/or phase offset
Measured gas properties (k and cv) are used either for direct signal compensation in thermal flow sensors and/or for determining a gas composition and pressure.

By specifically varying the excitation or excitation frequency, in accordance with embodiments, certain sensor geometries become selective for measuring quantities and insensitive to certain cross influences. This applies to different sensor geometries, but also to equal sensor geometries. Consequently, an embodiment provides a sensor system comprising a sensor cell, and an evaluation, as will be shown referring to FIG. 5.

FIG. 5 shows a sensor cell 10 comprising a heater 12, a cavity 14 and a substrate 16, in connection with evaluation 50. The evaluation 50 serves for driving the sensor cell 10 and is configured to drive the sensor cell 10 with at least two different excitation variations, like by means of two different excitation frequencies, for example. For example, the sensor which has a cutoff frequency for certain environmental conditions can be excited by a frequency which is significantly below the cutoff frequency, for example at half (½) the cutoff frequency or one third (⅓) or one fourth (¼) of the cutoff frequency (first measurement) and, for a second measurement, by an excitation frequency significantly above the cutoff frequency, for example two or three times the cutoff frequency (advantageously 3 to 20 times the cutoff frequency). This means that, generally, the first and second measurement which take place at different points in time, are different in that different excitation frequencies are used, advantageously an excitation frequency of smaller than ½ or smaller than ¼ of the cutoff frequency and/or an excitation frequency greater than three times the cutoff frequency. Thus, the sensor cell 10 can be operated at different operating points.

As can be seen from FIGS. 6a and 6b, operation at different operating points allows different sensitivities to result for heat conductivity and volume heat capacity. Consequently, the evaluation 50 is configured to determine the heat conductivity by means of the first measurement (operating point smaller than cutoff frequency) and to determine the volume heat capacity by means of the second measurement (operating point greater than cutoff frequency). Two exemplarily selected operating points or frequencies for the two measurements are illustrated in the diagrams of FIGS. 6a and 6b. This mode of operation is of advantage since the volume heat capacity and the heat conductivity can be determined independently of each other by using only a single sensor. The advantage in the variation of FIG. 3 is that the measurements take place simultaneously, whereas in FIG. 5 the measurements take place serially, that is at different points in time.

In accordance with embodiments, a sensor may exhibit sufficient insensitivities to cross-influences, for example, of the gas property not to be measured in the current measurement, for example, if operated in the first measurement in a first frequency range which is lower by about the factor of 4 than the cutoff frequency, and for a second measurement, in a second frequency range which is higher by about the factor of 4 than the cutoff frequency of the sensor system.

In accordance with embodiments, the excitation frequency for determining the heat conductivity may be below the cutoff frequency, like smaller than ¼ or smaller than ½. In accordance with further embodiments, the excitation frequency for determining the volume heat capacity may be above the cutoff frequency, like by about a factor of 3 to 20 above it or, generally, greater by a factor of 2 or 3. In these regions, the sensitivities Sₖ and S_{cv} are different. In accordance with embodiments, the cutoff frequency depends on the dimensions of the sensor or the implementation of the sensor. This means that the correlations between sensor dimensioning and selecting the operating point are to be applied while considering that the operating point, in accordance with embodiments, for the first and second measurements is to be selected to be different, depending on the cutoff frequency as discussed above.

In accordance with embodiments, irrespective of the dimensioning of the structures, high sensitivity to heat conductivity can be obtained at low frequencies. The frequency may, in accordance with embodiments, also be f=0, which corresponds to DC operation. This means that the excitation frequency is in a range of f≥0, that is, for example, close to zero. In accordance with embodiments, the excitation frequencies are different, i. e. are of different magnitudes. In accordance with embodiments, with high frequencies, the structure may be insensitive to heat conductivity (Sₖ approaching 0). In accordance with further embodiments, the sensitivity to volume heat capacity may comprise a local maximum.

$$\partial {S}_{cv} / \partial cv = 0$$

It is known from literature that a specified optimization (of both geometry and frequency) is not possible based on the parameter model. It follows that a suitable optimum for the operating point of the sensor arrangement has to be looked for.

With regard to determining the sensitivities to heat conductivity and volume heat capacity, reference is made to the above formula, discussed in connection with FIG. 3.

In accordance with further embodiments, instead of a variation of the excitation frequencies with two fixed frequencies, the sensor 10 can also be excited using a varying signal, like Dirac signal, for example, and then evaluation can take place at different frequencies where the corresponding sensitivities to heat conductivity and volume heat capacity form. The principle has been discussed in connection with FIG. 3 (see “Evaluation”), but can be transferred to the implementation of FIG. 5, in accordance with embodiments. Potential variations for an excitation signal were discussed in connection with FIG. 3 (see “Excitation”). As regards the dependency on the sensor dimensions, the dimensioning variations of FIG. 3 are to be referred to.

In accordance with embodiments, one of the sensors of FIGS. 2a-q may be used as sensor 10.

Applications for the sensor system of FIG. 5 will be discussed in connection with FIG. 9a and, in particular, in connection with FIGS. 9b-d. For example, the sensor system from FIG. 5 or the operating method for operating a sensor, as discussed in connection with FIG. 5, can be used for wide-range sensors, like wide-range pressure sensors. In addition, in accordance with further embodiments, the sensor system from FIG. 5 or the corresponding operating method may also be used for applications for flow measurements (compensated flow sensor) or for compensated pressure sensors.

With regard to excitation, it is also to be mentioned that, for example, alternatingly hopping between two frequencies is possible in order to achieve high sensitivity to heat conductivity and volume heat capacity (for example pure sine signal or using harmonics). The at least two different excitations are used for one, two or more sensors (depending on the setup from FIG. 3 or FIG. 5). The result is a modulated periodic excitation with both evaluation frequencies for heat conductivity and volume heat capacity. Potential periodic signal shapes, apart from a sine-shaped excitation, are square wave signals or saw tooth signals.

Different sensor cells will be discussed below referring to FIGS. 2a-o, which all can be employed in the examples mentioned above (embodiments or comparison examples).

FIG. 2a shows the known sensor 10 from FIGS. 1a and 1b having the heater 12 above the cavity 14. In FIG. 2b, a meandering heater 12′ above the cavity 14 is shown, wherein embodiments show that different dimensioning is achieved by different geometry variations of the heater 12′ since the heater 12′ is significantly longer than the heater 12. Both heaters 12 and 12′ are self-supporting structures or self-supporting bridge structures located above the cavity 14.

FIG. 2c also shows a self-supporting bridge structure, but with feed lines having an increased cross-section. This means that a temperature spot of the heater 12″ forms in the center. A similar temperature spot also forms in the heater 12′″ from FIG. 2d since the meander shape is arranged in particular in the center of the cavity. Here, the cavity 14′ is increased relative to the cavity 14 from FIG. 2b. All the previous variations of FIGS. 2a, 2b, 2c and 2d have in common that the cavity has a basically rectangular shape. However, this is not absolutely necessary as is shown, for example, in FIG. 2e.

FIG. 2e shows a sensor cell comprising a round cavity 14″ and a spiral-shaped heater 12″″, which has a two-dimensional appearance.

As mentioned before, the heaters comprise either the type of a self-supporting bridge structure, as is seen in FIG. 2a, FIG. 2c or FIG. 2d, for example. The heater exemplarily includes a conductive material which emits a corresponding Joule energy when a current flows through the same. The conductive material, like metal, for example, forms the self-supporting structure in this case. In accordance with further embodiments, it would also be conceivable for additional supporting structures to be provided, for example, by a membrane or perforated membrane. In accordance with embodiments, the self-supporting structure can be clamped on one side or two sides or, generally, several sides. A structure clamped on one side may also be referred to as a heating rib.

In accordance with embodiments, it would also be conceivable for the heating rib or, generally, the self-supporting structure to be perforated, as is shown in FIG. 2g in the structure 12″″. An increase in the perforated structure 12″″ is illustrated in FIG. 2f. As can be recognized, hexagonal openings are provided here.

In accordance with further embodiments, two heaters 12a and 12b may be arranged above a cavity 14, as can be recognized in FIG. 2h. The heaters may be equal or different. The heaters 12a and 12b illustrated here are parallel to each other and arranged at an equal spaced-apart height above the cavity 14 or heat sink at the bottom of the cavity 14. In accordance with further embodiments, it would also be conceivable for the two heaters 12a and 12b to cross above the cavity 14 so that the two heaters 12a and 12b are arranged at different heights.

In accordance with a further variation, a separate cavity 14a and 14b may be provided for each heater 12a and 12b, as is illustrated in FIG. 2j.

In the above embodiments, it was assumed that the cavity 14 or 14a or 14b or, more precisely, the bottom of the cavity serves as a heat sink. This means that the distance is decisive for the oscillation behavior so that the individual sensor cells can be dimensioned differently using this distance. In accordance with further embodiments, it would also be conceivable for an alternative or additional heat sink to be introduced apart from the heater, as is shown in FIG. 2k. Apart from the heater 12 arranged above the cavity 14, a further heat sink, for example made of metal, is provided, which here is provided with the reference numeral 13. Two heat sinks may also be formed by two substrates 16a and 16b enclosing the heater 12a. A cavity is formed between the two substrates 16a and 16b, where the heater 12 is positioned. Using a plurality of heat sinks 16a, 16c and 16d is shown in FIG. 2m. In this embodiment, a spacer layer 17 is applied on a substrate, which layer 17 comprises a recess below a heater 12 so that a cavity is formed below the heater 12. Laterally to the heater 12, a heat sink 16c1 and 16c2 is provided in the same level of the heater 12.

FIG. 2n shows a further variation. Several heaters 12* are applied here on the substrate surface of the substrate 16 as parallel structures/heating ribs spaced apart from a substrate 16. The heater of the substrate 12* is implemented as a heating rib having a foot point which is significantly widened relative to the heating rib. This is due to, in particular, manufacturing-technological factors. A further heater manufactured and implemented in a similar way is shown in FIG. 20. Here, the heating rib is again provided with the reference numeral 12*. The variations of FIGS. 2n and 20 are so-called surface micromechanics.

In summary, it can be stated that most different implementations can be used, like honeycomb structures, membrane (with/without holes), additional elements for active heat transmission to the measuring gas (like aluminum), meandering arrangements etc. The potential arrangement of an optional detector will be discussed below.

FIG. 2p shows the heater 12 in combination with the detector 18. The two elements are arranged next to each other in the same level, that is spaced apart equally, above the cavity 14.

FIG. 2q shows the heater 12 on which the detector 18 is arranged to be separated by means of an insulation layer. The insulation layer is provided with the reference numeral 18i. It is of advantage here that excitation and detection take place close to each other, wherein nevertheless excitation and detection are separate. The following detection variations exist, for example, in accordance with embodiments:
resistance detector is stacked above the heater and separated by insulation layer
resistance detector is arranged next to the heater (parallel, surrounding the heater,
thermal elements may be used in analogy to resistance detectors.

Alternatively, the heater itself can be used as detector, for example by evaluating an electrical response signal. This means that, in accordance with further embodiments, excitation and detection may be performed by the same element (Joule heating of the heater and resistive evaluation of the temperature signal). This variation is not illustrated here.

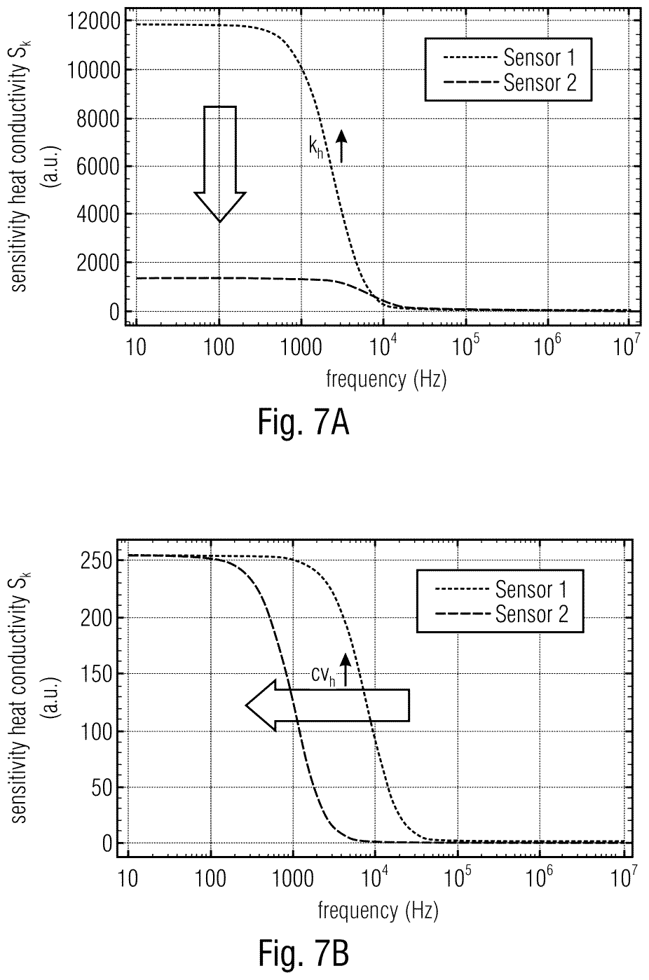

In FIG. 7, ways of changing the frequency-dependent sensitivity S_{K} using the material properties of the heating structure are shown.

Referring to FIG. 7a, which shows the sensitivity of kh over frequency and FIG. 7b, which shows the sensitivity of cvₕ over frequency for two different sensors each, the implementation of one or two sensors in an implementation in accordance with FIG. 3 or the implementation in accordance with FIG. 5 is discussed.

The effect of the heat conductivity of the sensor signal on the frequency-dependent sensitivity Sₖ is shown in FIG. 7a. The two sensors are of equal setup, but the heating structure of sensor 1 has lower a heat conductivity kₕ than sensor 2. The effect of the heat conductivity of the sensor signal on the frequency-dependent sensitivity S_{K} is shown in FIG. 7b. The two sensors are of equal setup, however, the heating structure of sensor 1 has lower a volume heat capacity cvₕ than sensor 2.

Sensor 1 and measurement M1 are to be sensitive to k, whereas sensor 2 and measurement M2 are to be insensitive to k. Two different solutions can be applied here, that is:
a) Solution for lower frequencies (f<<f_{cutoff} or f→0)
b) Solution for higher frequencies in the range of the cutoff frequency (0.5*f<f_{cutoff}).

The following three optimization ways result for a):
Optimize ratio of the cavities: d₁/d₂>20
Optimize ratio of heat conductivities of the heaters: kₕ₁/kₕ₂<0.05
Optimize ratio of product of layer thickness and width of the heaters: (b₁*h₁)/(b₂*h₂)<0.2 (precondition: equal heat transfer area to gas).

The following way of optimization results for solution b:
Optimize ratio of product of volume heat capacity and height of the heater:

$$( {cv}_{h 1} * {h}_{1} ) / ( {cv}_{h 2} * {h}_{2} ) < 0.25$$

It is to be pointed out here that the ways of optimization as mentioned above are each to be understood as specific embodiments so that further variations of optimization are conceivable in accordance with further embodiments.

In accordance with embodiments, a geometry adjustment to the gases may take place. The higher k_{gas}, the greater the cavity. Doubling k_{gas} results in quadruplicating d. The smaller k_{gas} and the higher cv_{gas}, the smaller can the frequency be selected.

Starting from the requirements that a sufficient sensitivity to cv is to be possible by means of sensor 1 or measurement M1, and that a sufficient insensitivity to cv is to be obtained by means of sensor 2 or measurement M2, the following solution can be selected for higher frequencies in the range of the cutoff frequency.

When combining the two applications while considering the teaching which can be recognized from FIGS. 7a and 7b, in accordance with embodiments, it is to be stated that the heat conductivity is advantageously evaluated below the cutoff frequency, whereas volume heat capacity is evaluated above the cutoff frequency.

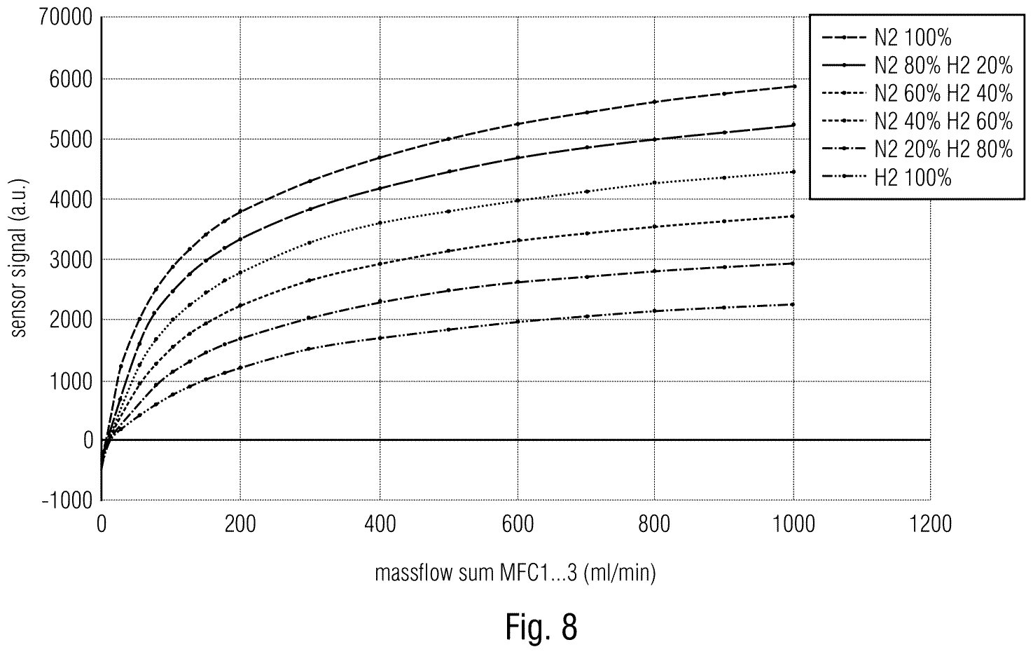

As mentioned already, the gas composition has an effect on all the measurements and, thus, on the output signal in thermal flow sensors, which can be seen from FIG. 8.

FIG. 8 shows a sensor signal as a function of a flow rate measured for different gas compositions. The result is a multitude of characteristic curves. This reveals the necessity for the relevant gas parameters to be advantageously established in direct proximity to the thermal flow sensor in order to determine the characteristic curve in flow sensors. The gas composition can easily and effectively be established using the variations from FIG. 3 or 5, while considering the above teaching.

These micro-technological sensors from FIGS. 3 and 5 can, in accordance with embodiments, be used either for signal compensation in thermal flow sensors, with changing gas media and operating parameters (pressure, temperature), but also offer a way for being used as individual sensors for determining the volume heat capacity, heat conductivity, temperature and pressure.

The result is the application of an inline-capable flow sensor offering a way for signal compensation. FIG. 9a shows a flow sensor 70 with the actual flow sensor 72 in combination with a sensor arrangement 1 comprising the two sensor chips 10a and 10b. The sensor 1 has several gas parameter-sensitive sensors and is located in a cavity of the chip 72, that is in a flow-restricted area. A thermal flow sensor having a membrane with holes for gas exchange is provided on the surface of the sensor 70.

The functionality, transferred to the variation of the sensor from FIG. 5, is illustrated schematically in FIG. 9b. FIG. 9b shows the two sensors 10 and 72. k of a known gas mixture 3 is determined by means of the sensor 10a (first measurement M1). In addition, ρ*c is determined for the same gas mixture 3 by the same sensor 10 (in the second measurement M2). These two parameters determined can be transferred for compensation to an evaluation device of the flow sensor 72, which determines a flow rate of the gas mixture 3. Compensation may, for example, take place by means of a lookup table so that a compensated flow rate is determined.

For a known gas mixture 3 (heat conductivity and volume heat capacity at reference temperature and reference pressure are known), the following method may apply:
Evaluation of the output signals (amplitude of temperature response) of measurement M1, which is proportional to the heat conductivity. The heat conductivity depends on the temperature and serves for determining the mean gas temperature.
Obtaining the output signal of sensor 10a from measurement M1 is used for compensating the output signals of sensor signals 10b (amplitude). The output signal of sensor 10b for measurement M2 depends on the volume heat capacity. The density can be determined by means of compensation, which serves for determining the pressure.

In accordance with further embodiments, the gas temperature T=f(k_{gas}) can be determined based on the sensor values of measurement M1, and the pressure p=f(cv_{gas}) by means of the sensor signal of measurement M2. In both variations, a lookup table can be used. It is to be pointed out here that it is not absolutely necessary for the gas mixture to be known (cf. FIG. 9c).

For example, using a further sensor 75, that is a temperature sensor, the gas composition can also be determined based on the sensor signal of measurement M1, based on an unknown mixture 3*. Knowing the gas composition vol. %=f(k_{gas}), using the sensor signal of measurement M2, a corrected flow rate can be determined, as discussed above (using the flow sensor 72).

As is shown in FIG. 9d, the temperature sensor is not absolutely necessary since determining the two values k and pc using the two measurements, based on the unknown gas mixture 3*, is sufficient to compensate the flow rate of the flow sensor 72. It is a peculiarity here that temperature, pressure and gas composition are not determined directly, wherein these are not absolutely necessary for absolute signal compensation.

The above discussion has shown that a further comparison example relates to a flow sensor having a sensor arrangement of FIG. 3. Here, the sensor 10a, for example, exhibits high sensitivity to heat conductivity and sensor 10b exhibits high sensitivity to volume heat capacity. In accordance with an embodiment, the sensor 10 with the evaluation 50 from FIG. 5 may also be used since the same parameters can be determined correspondingly during operation using two measurements (measurement M1 and measurement M2 at two different/successive points in time t1, t2).

A further embodiment relates to a pressure sensor which determines a compensated pressure, knowing the parameters k and ρ*c.

It is to be pointed out here that the heat conductivity is advantageously evaluated below the cutoff frequency, as can be recognized from FIGS. 7a and 7b.

A further embodiment relates to a method for operating the sensor arrangement. In accordance with embodiments, the operating point can be determined here. A method for determining the optimum sensor configuration may be implemented as follows:

In order to configure the sensor/sensor arrangement, for example for a universal measuring range, in accordance with embodiments, the operating points can be looked for using a self-adjusting method:
Sensor geometry: creating and varying the structure for operation at equal frequency
Sensor operation: frequency scan for determining the gas-dependent operating point (also dependent on pressure/temperature)→looking for the local maximum for the highest difference in sensitivity between sensor groups 1 and 2.

In order to determine the optimum operating points of the highest sensitivity to the measurement quantity (heat conductivity or volume heat capacity) for a sensor arrangement within the multi-dimensional parameter field, changes in the heat conductivity (like temperature variations, gas composition) and/or volume heat capacity (like pressure, gas composition) have to be provoked: this may be done on a calibration measuring station for a configured sensor arrangement. However, even in a non-optimum operating point, the measuring quantities can be determined, that is the sensor arrangement can also be used in a non-calibrated state.

In accordance with embodiments, the sensor chip, like the sensor chip of FIG. 5 or sensor chip of FIG. 3, comprises evaluation. Preferably, the one or more sensors are manufactured on a common chip. Depending on the compatibility with the manufacturing processes with thermal flow sensors, the flow sensor can be manufactured on the same chip. In accordance with a further embodiment, the ASIC can also be manufactured on the same chip. The ASIC or, in general, evaluation electronics is configured to easily process the combination of signals of several sensors for a dynamic on-chip signal compensation of thermal flow sensor. Thus, a highly miniaturized sensor of high dynamics and potential for monolithic integration is provided.

In accordance with embodiments, one or more sensors and one and/or more sensors in combination with evaluation are integrated monolithically. Here, either only the sensor for determining the gas parameters is provided, or the sensor may be extended by pressure sensors or flow sensors.

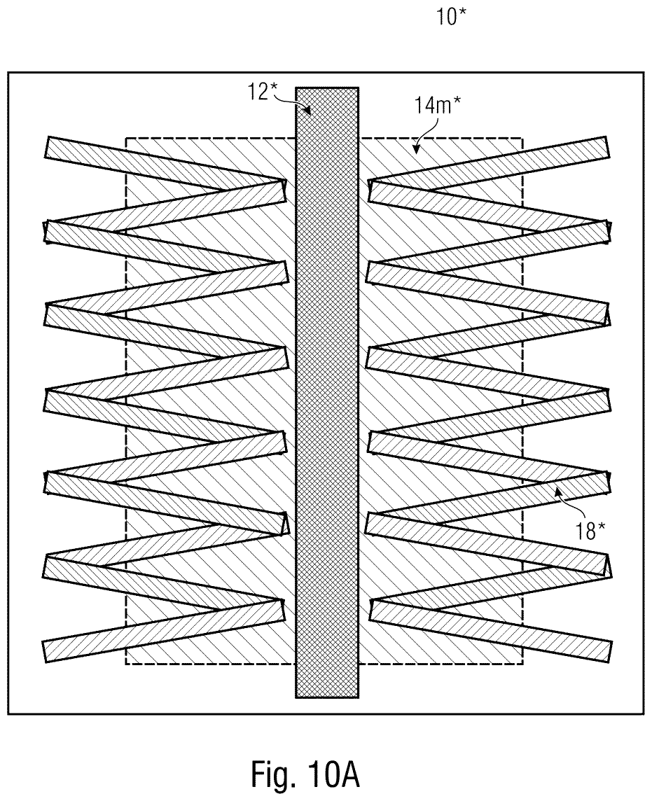

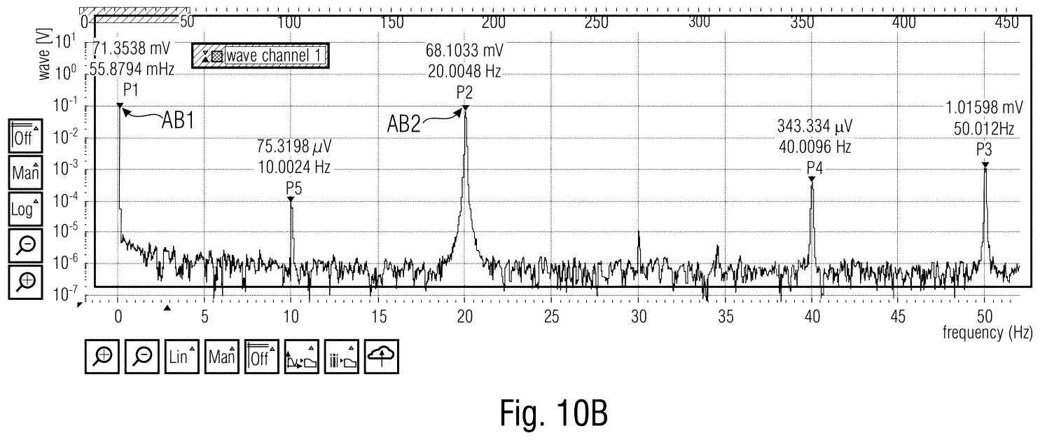

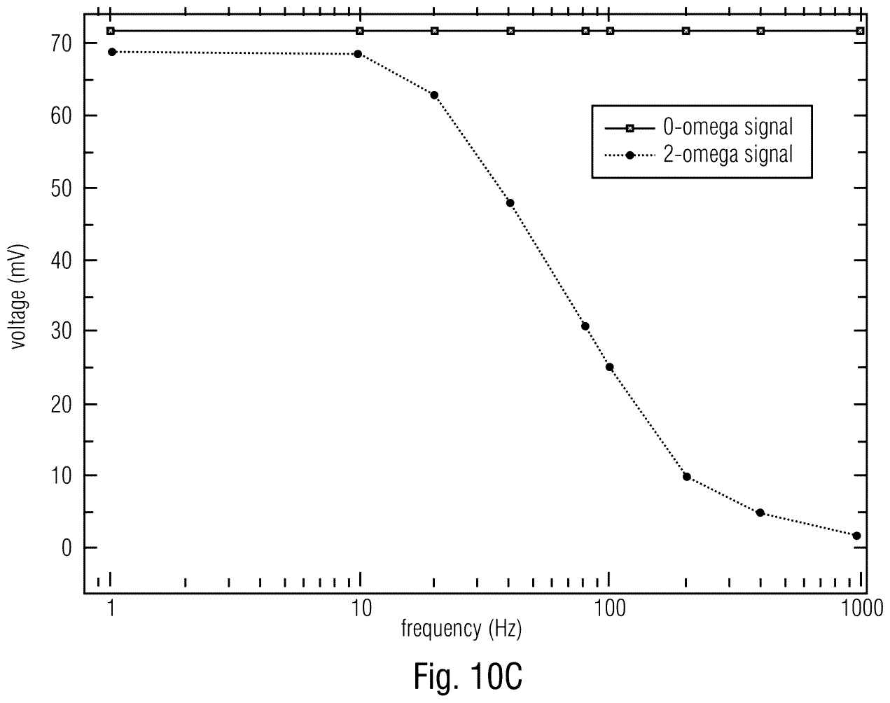

An extended embodiment in connection with evaluation will be discussed referring to FIGS. 10a, 10b, and 10c. FIG. 10a shows a layout of a potentially employable heating element 10*. It is to be pointed here that this layout, in accordance with embodiments, may also be applied to the above embodiments. FIG. 9b shows a diagram obtained by an FFT for illustrating the embodiment. FIG. 10c shows a diagram (voltage over frequency) for illustrating two evaluation signals or evaluation frequencies.

FIG. 10a shows a layout 10* having one or more heaters 12* (like metal wires) which may be arranged on an optional membrane 14m (which covers the cavity), and thermal elements 18* for temperature detection of the heating element 12*. A periodic current with a circular frequency Ω is fed in the metal wire: I(t)=I0×cos(Ωt). The thermal power fed in the heating wire equals: P(t) I²×R=I(t)²×R=(I0²×R/2)×(1+cos(2Ωt)), wherein R is the electrical resistance of the heater 12*. The temperature of the heater changes, due to the power fed, with the same frequency as the power signal: T(t)=T0(t)+ΔT×cos(2Ωt+ϕ). The amplitude ΔT and the phase shift ϕ relative to the feeding power depend on the heat conductivity of the material and the frequency Ω. T0 is the zero position of the temperature oscillation and depends on the power and coupling between the sample and the environment. The temperature oscillation of the heating wire results in a resistance oscillation thereof. This discussion of coupling or causing thermal oscillation is to be applied to the above embodiments.

Starting from an oscillation thermally excited in this way, the heating element, here heating element 12*, can be excited by only one frequency (like 1 kHz). In this embodiment, the evaluation frequency is evaluated at two points, for example, at the 0-th and the 2nd harmonic. The 0-th harmonic oscillation is also referred to as DC signal. The 2nd is referred to as 2-omega signal. This evaluation by means of an FFT analysis is illustrated in FIG. 10b. In FFT analysis, different harmonic oscillations form. Of interest are, above all, the 0-omega signal and the 2-omega signal, which here are referred to by AB1 and AB2. AB1 represents the 0-omega signal or DC signal and is used as a measure of T0. AB2 represents the 2-omega signal and is used as a measure of ΔT. What results is the temperature signal T(t)=T0 (t)+ΔT×cos(2Ωt+ϕ). In accordance with further embodiments, another harmonic signal or other frequency signal may be used, like the 1-omega signal, for example. It is to be pointed out here that in the embodiments illustrated in FIG. 10b, an excitation of 10 Hz was used. In this embodiment, the evaluation frequencies are 0 Hz and 20 Hz.

In FIG. 10c, the 0-omega signal and the 2-omega signal over the frequency range are illustrated. Starting from this diagram, the 0-omega signal (DC signal) can be used as a measure of heat conductivity, whereas the 2-omega signal, with higher frequencies, represents a measure of temperature conductivity and volume heat capacity.

This means that the above embodiment has shown that a 0 Hz frequency may also be used as evaluation frequency when two evaluation frequencies are to be used. This procedure has proven to be useful for first measurements in order to achieve, with only a single excitation frequency, high sensitivity to heat conductivity (DC component) and high sensitivity to volume heat capacity.

In accordance with an embodiment, the evaluation with the two evaluation frequencies may take place by means of an FFT analysis. In accordance with embodiments, the FFT analysis of the harmonic temperature signals (like 2× excitation frequency and Ox excitation frequency (DC signal) may be performed). In accordance with a further embodiment, the detector measures the DC signal, whereas a further detector measures the harmonic signals. A plurality of detectors in connection with a heating element are illustrated in FIG. 10a, wherein this only represents an exemplary case.

A comparison example provides a sensor arrangement comprising at least two highly miniaturized sensors with thermal operating principles for determining an individual gas property (volume heat capacity cv (product of density and specific heat capacity) or heat conductivity k). These thermal sensors are configured such that at least one component exhibits high sensitivity to one gas property, whereas at least one further component exhibits high sensitivity to another gas property. The challenge is producing a structure, sensitive to a gas property, while at the same time minimizing cross-sensitivities. As mentioned already, the gas properties are not only dependent on the composition, but also temperature and pressure. However, this influence varies strongly and can be used to employ several combined gas property sensors indirectly for determining pressure and temperature. Small changes in pressure (Δp<10 bar) in a first approximation only result in a change in the gas density. Variations in temperature (ΔT<50 K), however, in a first approximation, have an effect on density and heat conductivity. The specific heat capacity, however, remains almost uninfluenced by changes in pressure and temperature.

### Description of Variables Used Above:

dimensions of the heater: L, b, h
height of the cavity: d
material properties of the heater: cvₕ, kₕ
gas properties k, cv or, for improved unambiguity, k_{gas}, cv_{gas}
sensitivities to gas properties Sₖ, S_{cv}
width of the effective heat transfer area to gas b_{gas}
cutoff frequencies f_{cutoff} or f_{cutoff,S1}

Although some aspects have been described in the context of an apparatus, it is understood that these aspects also represent a description of the corresponding method so that a block or a structural component of an apparatus is also to be understood to be a corresponding method step or feature of a method step. In analogy, aspects described in the context of or as a method step also represent a description of a corresponding block or detail or feature of a corresponding apparatus. Some or all of the method steps may be performed by a hardware apparatus (or using a hardware apparatus), such as a microprocessor, a programmable computer or an electronic circuit. In some embodiments, some or several of the most important method steps may be performed by such an apparatus.

Depending on specific implementation requirements, embodiments of the invention may be implemented in hardware or in software. Implementation may be effected while using a digital storage medium, for example a floppy disc, DVD, Blu-ray disc, CD, ROM, PROM, EPROM, EEPROM or FLASH memory, a hard disc or any other magnetic or optical memory which has electronically readable control signals stored thereon which may cooperate, or cooperate, with a programmable computer system such that the respective method is performed. This is why the digital storage medium may be computer-readable.

Some embodiments in accordance with the invention thus comprise a data carrier which comprises electronically readable control signals which are capable of cooperating with a programmable computer system such that any of the methods described herein is performed.

Generally, embodiments of the present invention may be implemented as a computer program product having a program code, the program code being effective to perform any of the methods when the computer program product runs on a computer.

The program code may also be stored on a machine-readable carrier, for example.

Other embodiments include the computer program for performing any of the methods described therein, the computer program being stored on a machine-readable carrier. In other words, an embodiment of the inventive method thus is a computer program which has program code for performing any of the methods described herein, when the computer program runs on a computer.

A further embodiment of the inventive methods thus is a data carrier (or a digital storage medium or a computer-readable medium) on which the computer program for performing any of the methods described herein is recorded.

A further embodiment of the inventive method thus is a data stream or a sequence of signals representing the computer program for performing any of the methods described herein. The data stream or the sequence of signals may be configured, for example, to be transferred via a data communication link, for example via the Internet.

A further embodiment includes processing means, for example a computer or a programmable logic device, configured or adapted to perform any of the methods described herein.

A further embodiment includes a computer on which the computer program for performing any of the methods described herein is installed.

A further embodiment in accordance with the invention comprises an apparatus or a system configured to transmit a computer program for performing at least one of the methods described herein to a receiver. The transmission may, for example, be electronic or optical. The receiver may, for example, be a computer, mobile device, memory device or a similar apparatus. The apparatus or system may, for example, comprise a file server for transmitting the computer program to the receiver.

In some embodiments, a programmable logic device (for example field-programmable gate array, FPGA) may be used to perform some or all of the functionalities of the methods described herein. In some embodiments, a field-programmable gate array may cooperate with a microprocessor so as to perform any of the methods described herein. Alternatively, a microcontroller (like PSoC, Programmable System on Chip) and/or lock-in technology may be used. In general, in some embodiments, the methods are performed by any hardware apparatus, which may be universally employable hardware, like a computer processor (CPU), or hardware specific for the method, like an ASIC, for example.

While this invention has been described in terms of several embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations and equivalents as fall within the true spirit and scope of the present invention.

1. A sensor arrangement comprising at least one sensor cell and an evaluation,
wherein the at least one sensor cell can be excited thermally by means of a heater;
wherein the sensor cell is configured to form an oscillation behavior in dependence on a gas property of a gas surrounding the sensor cell, in particular a heat conductivity and/or volume heat capacity and/or temperature and/or pressure,
wherein the sensor cell is excited by means of at least one excitation frequency, and wherein a first excitation frequency or a first evaluation frequency is used for a first measurement, and wherein a second excitation frequency or evaluation frequency is used for a second measurement,
wherein the first excitation frequency differs from the second excitation frequency, or wherein the first evaluation frequency differs from the second evaluation frequency; and
wherein the evaluation is configured to determine heat conductivity of the surrounding gas based on the first measurement and volume heat capacity of the surrounding gas based on the second measurement,
wherein the first excitation frequency is smaller by at least a factor of 2 or at least a factor of 4 than the cutoff frequency of the sensor, and wherein the second excitation frequency is greater by at least a factor of 2 or at least a factor of 4 than the cutoff frequency of the sensor; and/or wherein the first evaluation frequency is smaller by at least a factor of 2 or at least a factor of 4 than the cutoff frequency of the sensor, and wherein the second evaluation frequency is greater by at least a factor of 2 or at least a factor of 4 than the cutoff frequency of the sensor.

2. The sensor arrangement in accordance with claim 1, wherein the first excitation frequency and/or the second excitation frequency are greater than or equaling 0 Hz;
wherein the first excitation frequency equals 0 Hz and wherein the excitation energy for the first measurement is greater than 0, or wherein the second excitation frequency equals 0 Hz and wherein the excitation energy for the second measurement is greater than 0.

3. The sensor arrangement in accordance with claim 1, wherein the first excitation frequency differs from the second excitation frequency by at least a factor of 2, at least a factor of 4 or at least a factor of 8; and/or
wherein the first and the second evaluation frequency differ by at least a factor of 2, at least a factor of 4 or at least a factor of 8.

4. The sensor arrangement in accordance with claim 1, wherein the first excitation frequency or the first evaluation frequency and the second excitation frequency or second evaluation frequency are each defined by a fixed frequency.

5. The sensor arrangement in accordance with claim 1, wherein the sensor cell comprises a cavity comprising a heater, or a heat sink comprising a spaced-apart heater, or a heating rib spaced apart from a heat sink; and/or
wherein the heater or heating rib is configured to oscillate thermally and thus form the oscillation behavior; and/or
wherein the heater is formed by a heating rib or a self-supporting structure or self-supporting bridge structure.

6. The sensor arrangement in accordance with claim 1, wherein the sensor cell comprises a detector configured to detect the oscillation behavior.

7. The sensor arrangement in accordance with claim 1, wherein the sensitivity to volume heat capacity in the second measurement is higher by at least a factor of 3, at least a factor of 4 or at least a factor of 5 than the sensitivity to volume heat capacity in the first measurement; and/or
wherein the sensitivity to heat conductivity in the first measurement is higher by at least a factor of 1.1 or at least a factor of 1.2 than the sensitivity to heat conductivity in the second measurement.

8. The sensor arrangement in accordance with claim 1, wherein the evaluation is configured to periodically excite the sensor cell.

9. The sensor arrangement in accordance with claim 1 wherein the sensor cell is excited by a varying excitation frequency, in particular a CHIRP signal or DIRAC signal.

10. The sensor arrangement in accordance with claim 1, wherein the evaluation determines the oscillation behavior of the sensor using the dynamic temperature response and/or using the amplitude and/or using the frequency and/or using the phase.

11. The sensor arrangement in accordance with claim 1, wherein the evaluation is implemented as an ASIC, wherein the ASIC is integrated in a chip or monolithic chip which accommodates the sensor cell.

12. The sensor arrangement in accordance with claim 1, wherein the first evaluation frequency and/or the second evaluation frequency are greater than or equaling 0 Hz; and/or
wherein the evaluation comprises an FFT; or
wherein the evaluation comprises an FFT and the first measurement takes place at a first evaluation frequency equaling 0 Hz and/or the second evaluation frequency is greater than or equaling 0 Hz or at 1-OMEGA, 2-OMEGA or 3-OMEGA.

13. A flow sensor comprising a sensor arrangement in accordance with claim 1, wherein the flow sensor is configured to determine a flow while considering the heat conductivity and volume heat capacity determined.

14. A pressure sensor comprising a sensor arrangement in accordance with claim 1, wherein the pressure sensor is configured to determine the pressure while considering the volume heat capacity and heat conductivity.

15. A method for evaluating a sensor arrangement in accordance with claim 1, comprising:
exciting the sensor by means of an excitation frequency, wherein a first excitation frequency or a first evaluation frequency is used for a first measurement, and wherein a second excitation frequency or evaluation frequency is used for a second measurement, wherein the first excitation frequency differs from the second excitation frequency, or wherein the first evaluation frequency differs from the second evaluation frequency; and
determining heat conductivity based on the first measurement and volume heat capacity based on the second measurement;
wherein the first excitation frequency is smaller by at least a factor of 2 or at least a factor of 4 than the cutoff frequency of the sensor, and wherein the second excitation frequency is greater by at least a factor of 2 or at least a factor of 4 than the cutoff frequency of the sensor; and/or wherein the first evaluation frequency is smaller by at least a factor of 2 or at least a factor of 4 than the cutoff frequency of the sensor, and wherein the second evaluation frequency is greater by at least a factor of 2 or at least a factor of 4 than the cutoff frequency of the sensor.

16. A non-transitory digital storage medium having stored thereon a computer program for performing a method for evaluating a sensor arrangement in accordance with claim 1, comprising: exciting the sensor by means of an excitation frequency, wherein a first excitation frequency or a first evaluation frequency is used for a first measurement, and wherein a second excitation frequency or evaluation frequency is used for a second measurement, wherein the first excitation frequency differs from the second excitation frequency, or wherein the first evaluation frequency differs from the second evaluation frequency; and determining heat conductivity based on the first measurement and volume heat capacity based on the second measurement; wherein the first excitation frequency is smaller by at least a factor of 2 or at least a factor of 4 than the cutoff frequency of the sensor, and wherein the second excitation frequency is greater by at least a factor of 2 or at least a factor of 4 than the cutoff frequency of the sensor; and/or wherein the first evaluation frequency is smaller by at least a factor of 2 or at least a factor of 4 than the cutoff frequency of the sensor, and wherein the second evaluation frequency is greater by at least a factor of 2 or at least a factor of 4 than the cutoff frequency of the sensor, hen the computer program is run by a computer or the evaluation.
